SEQUENCE LISTING (45 sequences)

SEQ ID NO: 1 - protein, 29 aa, synthetic construct
  MOD_RES 1: note = Acetylation
  MOD_RES 2: note = D-Phe
  MOD_RES 29: note = Amidation
YFDAIFTNSYRKVLGQLSARKLLQDIMSR

SEQ ID NO: 2 - protein, 23 aa, synthetic construct
  MOD_RES 23: note = Amidation
FTDNYTRLRKQMAVKKYLNSILN

SEQ ID NO: 3 - protein, 28 aa, synthetic construct
  MOD_RES 6: note = D-p-Cl-Phe
  MOD_RES 28: note = Amidation
HSDAVFTDNYTRLRKQLAVKKYLNSILN

SEQ ID NO: 4 - protein, 28 aa, synthetic construct
KPRRPYTDNYTRLRKQMAVKKYLNSILN

SEQ ID NO: 5 - protein, 6 aa, synthetic construct
KPRRPY

SEQ ID NO: 6 - protein, 22 aa, synthetic construct
TDNYTRLRKQMAVKKYLNSILN

SEQ ID NO: 7 - protein, 29 aa, synthetic construct
  MOD_RES 17: note = N-stearyl-Nle
KPRRPYTDNYTRLRKQLMAVKKYLNSILN

SEQ ID NO: 8 - protein, 27 aa, synthetic construct
  MOD_RES 1: note = Acetylation
  MOD_RES 2: note = D-Phe
  MOD_RES 27: note = Amidation
HFDAVFTNSYRKVLKRLSARKLLQDIL

SEQ ID NO: 9 - protein, 7 aa, synthetic construct
HFDAVFT

SEQ ID NO: 10 - protein, 20 aa, synthetic construct
NSYRKVLKRLSARKLLQDIL

SEQ ID NO: 11 - protein, 28 aa, synthetic construct
  MOD_RES 1: note = Acetylation
  MOD_RES 2: note = D-Phe
  MOD_RES 28: note = Amidation
HFDAVFTNSYRKVLKRLSARKLLQDILC

SEQ ID NO: 12 - protein, 28 aa, synthetic construct
  MOD_RES 1: note = Acetylation
  MOD_RES 2: note = D-Phe
  MOD_RES 28: note = Amidation
HFDAVFTNSYRKVLKRLSVRKLLQDILC

SEQ ID NO: 13 - protein, 27 aa, synthetic construct
  MOD_RES 1: note = Acetylation
  MOD_RES 2: note = D-Phe
  MOD_RES 27: note = Amidation
HFDAVFTNSYRKVLKRLSARKLLQSIL

SEQ ID NO: 14 - RNA, 20 nt, synthetic construct
  source 1..20: mol_type = other RNA
aagtgggataagagttcatc

SEQ ID NO: 15 - DNA, 120 bp, synthetic construct
  source 1..120: mol_type = other DNA
ggtttttgtaggggacaatttagaagtgggataagagttcataacttcgtataatgtatgctatacgaagttatggatccatctgggcctaggatgggttatagcctgggttggggttgg

SEQ ID NO: 16 - DNA, 25 bp, synthetic construct
  source 1..25: mol_type = other DNA
ggagccaagagtcctgagaaggccc

SEQ ID NO: 17 - DNA, 25 bp, synthetic construct
  source 1..25: mol_type = other DNA
cacaggctttcggagtagggggcca

SEQ ID NO: 18 - RNA, 20 nt, synthetic construct
  source 1..20: mol_type = other RNA
agccacagctagacccttaa

SEQ ID NO: 19 - DNA, 25 bp, synthetic construct
  source 1..25: mol_type = other DNA
ccctcacgtcacgagcccagtccaa

SEQ ID NO: 20 - DNA, 25 bp, synthetic construct
  source 1..25: mol_type = other DNA
tttgtgctgatgggctgctgcaggg

SEQ ID NO: 21 - DNA, 120 bp, synthetic construct
  source 1..120: mol_type = other DNA
cctccctccttgggtagcccagcagccacagctagaccctataacttcgtatagcatacattatacgaagttatggatcctaaaggtagtttccagataagagctgggaactccccagat

SEQ ID NO: 22 - DNA, 30 bp, synthetic construct
  source 1..30: mol_type = other DNA
tagtggatcccccggatgcgccctccgagc

SEQ ID NO: 23 - DNA, 36 bp, synthetic construct
  source 1..36: mol_type = other DNA
cgaggttaacgaatttcagaccagggagacctccgc

SEQ ID NO: 24 - DNA, 37 bp, synthetic construct
  source 1..37: mol_type = other DNA
cttaagaaaggtcgaccaccatgcgccctccgagcct

SEQ ID NO: 25 - DNA, 34 bp, synthetic construct
  source 1..34: mol_type = other DNA
tgccctctccggatccgaccagggagacctccgc

SEQ ID NO: 26 - DNA, 38 bp, synthetic construct
  source 1..38: mol_type = other DNA
cttaagaaaggtcgaccaccatggtgagcaagggcgag

SEQ ID NO: 27 - DNA, 37 bp, synthetic construct
  source 1..37: mol_type = other DNA
ccgctatcacagatcactagtcttgtacagctcgtcc

SEQ ID NO: 28 - DNA, 22 bp, synthetic construct
  source 1..22: mol_type = other DNA
agtatggatgagcagcaacaga

SEQ ID NO: 29 - DNA, 20 bp, synthetic construct
  source 1..20: mol_type = other DNA
agatagccatggcaaccagg

SEQ ID NO: 30 - DNA, 20 bp, synthetic construct
  source 1..20: mol_type = other DNA
actgtgacatccccaccaac

SEQ ID NO: 31 - DNA, 20 bp, synthetic construct
  source 1..20: mol_type = other DNA
gagtctaaggcggacaccag

SEQ ID NO: 32 - DNA, 24 bp, synthetic construct
  source 1..24: mol_type = other DNA
ctacggcaagctgaccctgaagtt

SEQ ID NO: 33 - DNA, 21 bp, synthetic construct
  source 1..21: mol_type = other DNA
ctcggcgcgggtcttgtagtt

SEQ ID NO: 34 - DNA, 20 bp, synthetic construct
  source 1..20: mol_type = other DNA
cgcttcggcagcacatatac

SEQ ID NO: 35 - DNA, 20 bp, synthetic construct
  source 1..20: mol_type = other DNA
ttcacgaatttgcgtgtcat

SEQ ID NO: 36 - RNA, 20 nt, synthetic construct
  source 1..20: mol_type = other RNA
ttgaacgggtcgaggtgcca

SEQ ID NO: 37 - RNA, 20 nt, synthetic construct
  source 1..20: mol_type = other RNA
aatggcggcgtcggcggcgt

SEQ ID NO: 38 - DNA, 17 bp, synthetic construct
  source 1..17: mol_type = other DNA
ccccgaagcgtgctcgt

SEQ ID NO: 39 - DNA, 20 bp, synthetic construct
  source 1..20: mol_type = other DNA
gcggctctttgctgtctctt

SEQ ID NO: 40 - DNA, 20 bp, synthetic construct
  source 1..20: mol_type = other DNA
actgaccagcacaccaacaa

SEQ ID NO: 41 - DNA, 20 bp, synthetic construct
  source 1..20: mol_type = other DNA
gctcaacggtggaaagagga

SEQ ID NO: 42 - DNA, 25 bp, synthetic construct
  source 1..25: mol_type = other DNA
ggggccgtttctcttgcaggctagc

SEQ ID NO: 43 - DNA, 25 bp, synthetic construct
  source 1..25: mol_type = other DNA
tctttctctttggggctgggctggg

SEQ ID NO: 44 - DNA, 25 bp, synthetic construct
  source 1..25: mol_type = other DNA
aaaaaggagggcgagtggcgagcag

SEQ ID NO: 45 - DNA, 25 bp, synthetic construct
  source 1..25: mol_type = other DNA
ccccaccctcacacctctggtcctg

(19) United States
(12) Patent Application Publication
(10) Pub. No.: US 2025/0161414 A1
(43) Pub. Date: May 22, 2025

(54) METHOD FOR IMPROVING AUDITORY PERCEPTION USING A VIPR1 INHIBITOR

(71) Applicant: ST. JUDE CHILDREN'S RESEARCH HOSPITAL, INC., Memphis, TN (US)

(72) Inventors: Stanislav ZAKHARENKO, Memphis, TN (US); Christopher M. DAVENPORT, Memphis, TN (US)

(21) Appl. No.: 18/840,184

(22) PCT Filed: Feb. 21, 2023
(86) PCT No.: PCT/US2023/062925

§ 371 Date: Aug. 21, 2024

Related U.S. Application Data
(60) Provisional application No. 63/312,555, filed on Feb. 22, 2022.

(51) Int. Cl. A61K 38/22; C12N 15/113

(52) U.S. Cl. CPC A61K 38/2278; C12N 15/1138; C12N 2310/14

(57) ABSTRACT

Methods for increasing auditory cortex interneuron excitability and improving auditory perception, in particular pitch-discrimination acuity, are provided, which include the use of a Vasoactive Intestinal Peptide Receptor 1 (VIPR1) inhibitor.

### REFERENCE TO RELATED APPLICATIONS

This application claims benefit of U.S. Provisional Patent Application Ser. No. 63/312,555, filed Feb. 22, 2022, the content of which is incorporated herein by reference in its entirety.

### REFERENCE TO AN ELECTRONIC SEQUENCE LISTING

The contents of the electronic sequence listing (name: SJ0103WO_ST26. xml; size: 47,828 bytes; and date of creation: Jan. 9, 2023) is herein incorporated by reference in its entirety.

### BACKGROUND

The ability to distinguish one pitch (the auditory perception associated with the frequency of a sound) from another or from the surrounding acoustic background is essential for everyday hearing, linguistic abilities, and musicality. For instance, musicality, which spans from congenital amusia, to relative pitch, and to absolute (perfect) pitch depends on the ability of the auditory system to faithfully identify or separate sounds with adjacent frequencies. Musicality is either innate or acquired by musical training. A high degree of heritability of musicality or perfect pitch has been suggested by aggregation of highly musical individuals in some families and twin studies. The mechanisms of innate musicality and the underlying enhanced pitch-discrimination acuity are mostly unknown.

Williams-Beuren syndrome (WBS) is a rare neurodevelopmental disorder caused, in most cases, by 1.55- to 1.83-Mb hemizygous microdeletions containing 25-27 contiguous genes in chromosomal locus 7q11.23. Music and language capabilities of individuals with WBS are relatively preserved or even enhanced, despite developmental delays, intellectual disability (average IQ <70), and other significant cognitive and learning deficits. WBS is associated with greater emotional response to certain sounds, particularly music, with the love of and interest in music being noted among the earliest descriptions of patients with WBS. More recent work has described enhanced musical abilities and skills, remarkable auditory acuity, and a prevalence of “perfect pitch” among persons with WBS.

Pitch perception and discrimination are attributed in part to the neurophysiology of the auditory cortex (ACx). Human subjects with WBS have atypical structural and functional neuroanatomy related to auditory processing. Despite lower overall volume of the cortex, that of the ACx is either spared or increased. Furthermore, individuals with WBS have elevated auditory-evoked potentials and atypical activation of cortical areas by sound, suggesting that ACx abnormalities underlie WBS hyperacuity or other auditory enhancements in WBS. Indeed, perception of music and speech is attributed to higher-order cortical areas, including the ACx. Studies in animals suggest that the ACx is involved in acoustic-frequency discrimination. Specifically, activity of inhibitory neurons in the ACx contributes to innate frequency-discrimination acuity. Optogenetic activation or inhibition of parvalbumin-positive (PV⁺) GABAergic interneurons in the ACx improved or worsened, respectively, the behaviors that rely on innate frequency discrimination.

### SUMMARY OF THE INVENTION

This invention provides methods for increasing auditory cortex interneuron excitability and improving auditory perception (e.g., pitch-discrimination acuity) in a subject by inhibiting the expression or activity of Vasoactive Intestinal Peptide Receptor 1 (VIPR1). In some aspects, VIPR1 activity is inhibited with a VIPR1 peptide antagonist. In other aspects, VIPR1 expression is inhibited with a VIPR1 inhibitory RNA molecule. Subjects benefiting from the use of a VIPR1 inhibitor include those with amusia, as well as musicians and linguists.

### DETAILED DESCRIPTION

Pitch-discrimination acuity is mediated by VIPR1 (Vasoactive Intestinal Peptide Receptor 1), the inhibition of which induces hyperexcitability of GABAergic interneurons (but not excitatory neurons) in the ACx and improves frequency coding by the ACx. Accordingly, this invention provides methods for increasing auditory cortex interneuron excitability and improving auditory perception in an otherwise healthy ear by inhibiting or decreasing VIPR1 expression or activity in auditory cortex interneurons. Manipulating VIPR1 expression or function finds use in improving auditory capabilities in humans (e.g., musical professionals) and/or treating amusia (tone deafness), which affects up to 4% of population.

As is known in the art, the auditory cortex (ACx) is composed of many different types of neurons, including excitatory neurons, inhibitory neurons and interneurons, the latter of which form reciprocal connections not only with the excitatory neurons, but also with each other. In accordance with the methods f this invention, increasing the excitability of interneurons (preferably GABAergic interneurons, most preferably parvalbumin-positive PV⁺ interneurons) improves auditory perception, in particular in the form of pitch-discrimination acuity.

Pitch is one of the primary auditory sensations, along with loudness and timbre. Pitch refers to the specific frequency of a sound wave, which is measured in Hertz (Hz). A high frequency (e.g., 880 hertz) is perceived as a high pitch and a low frequency (e.g., 55 Hz) as a low pitch. In music, sequences of pitch define melody, and simultaneous combinations of pitch define harmony. In speech, rising and falling pitch contours help define prosody and in tone languages, such as Mandarin and Cantonese, pitch contours help define the meaning of words. In complex acoustic environments, differences in pitch can help listeners to segregate and make sense of competing sound sources. The capacity to recognize modifications in noise based upon pitch is referred to as pitch-discrimination acuity or frequency discrimination. Assessments of pitch-discrimination acuity, and improvements in the same, can be determined by the methods described herein, i.e., pre-pulse inhibition (PPI) of the acoustic startle response or other conventional methods such as frequency following response (FFR), also referred to as frequency following potential (FFP) or envelope following response (EFR). An improvement or enhancement in pitch-discrimination acuity and auditory perception refers to a subject's ability to distinguish between two closely related pitches after treatment with a VIPR1 inhibitor as compared to before treatment. By way of illustration, pitch-discrimination acuity is improved/enhanced when a subject before treatment can only perceive a difference between sounds that differ by 100 hertz, whereas after treatment the subject can distinguish between sounds that differ by 50 hertz.

Human Vasoactive Intestinal Peptide Receptor 1 (VIPR1 or VPAC₁ receptor) is known in the art as a membrane-bound receptor having the nucleotide and protein sequences available, e.g., under GENBANK Accession Nos. NM 001251882 and NP 004615, respectively. In accordance with the methods of this invention, the expression or activity of VIPR1 is inhibited using a VIPR1 inhibitor. The term “VIPR1 inhibitor” is not intended to embrace non-selective suppressors of all gene expression or protein synthesis, or general toxins.

In one aspect, the activity of VIPR1 is inhibited using a VIPR1 receptor antagonist (or simply “VIPR1 antagonist”). A VIPR1 antagonist is any molecular species that interferes with normal VIPR1 receptor activity. The VIPR1 antagonist can be a VIPR1 receptor binding molecule that interferes with the interaction between PACAP and VIPR1. For instance, suitable antagonists include small molecules, peptides or non-peptides which structurally mimic the natural substrates of VIPR1, but which do not activate the receptor. VIPR1 antagonists also include molecular species which do not mimic the natural substrates of VIPR1, but which interact with the substrate binding domain or other regions of the receptor, preventing its activity. In certain aspects, suitable antagonists include short peptides or antibodies, including fragments of antibodies, such as Fc, which selectively bind to and inhibit the activity of the VIPR1 (collectively referred to herein as “VIPR1 peptide antagonists”).

The activity of VIPR1 receptor can be assayed by measuring changes in intracellular CAMP or inositol-3-phosphate concentrations. By way of illustration, VIPR1 activity can be assayed by incubating cells that express VIPR1 in medium (e.g., DMEM, HANKS) in the presence of an antagonist at a temperature of about 37° C. for several minutes (e.g., 10 minutes). The time course of production of CAMP, inositol-3-phosphate and Ca²⁺ can be detected by measuring methods well known in the art (Masmoudi et al. (2003) FASEB 17:17-27). Assays for measuring the production of CAMP, inositol-3-phosphate metabolism and Ca²⁺ cellular concentration have been described in the art.

In certain aspects, the VIPR1 antagonist is selective for VIPR1. A VIPR1 selective antagonist refers to a compound that is able to selectively bind to VIPR1 and reduce VIPR1 activation by an agonist. VIPR1 selective antagonists will bind to the VIPR1 at significantly lower concentrations than the VIPR2 receptor. Selectivity is determined by comparing the IC₅₀'s of the receptor antagonist for the VIPR1 and VIPR2 receptors. Typically, the selectivity for the VIPR1 will be at least about 2:1, preferably at least about 10:1, more preferably at least about 100:1 and most preferably at least 1000:1 over the VIPR2 receptor. The lower the IC₅₀ of a receptor antagonist relative to its IC₅₀ for other receptors, the greater the selectivity.

A number of VIPR1 antagonists are disclosed in U.S. Pat. Nos. 5,565,424; 7,094,755; 6,828,304; WO 2019/094817 and WO 2005/089229. In particular aspects, the VIPR1 is a peptide antagonist. Examples of suitable VIPR1 peptide antagonists include, but are not limited to:
[Acetyl-Tyr¹, D-Phe²] GRF 1-29 with C-terminal amidation, i.e., Ac-YfDAIFTNSYRKVLGOLSARKLLODIMSR* (SEQ ID NO:1);
VIP (6-28) with C-terminal amidation, i.e., FTDNYTRLRKQMAVKKYLNSILN* (SEQ ID NO:2);
[D-p-Cl-Phe⁶, Leu¹⁷]-VIP with C-terminal amidation, i.e., HSDAVETDNYTRLRKOLAVKKYLNSILN* (SEQ ID NO: 3);
neurotensin-(6-11)/VIP-(7-28) hybrid, i.e., KPRRPYTDNYTRLRKQMAVKKYLNSILN (SEQ ID NO:4), a hybrid peptide of neurotensin residues 6-11 (KPRRPY; SEQ ID NO:5) and VIP residues 7-28 (TDNYTRLRKQMAVKKYLNSILN; SEQ ID NO:6);
N-stearyl-[Nle¹⁷] neurotensin-(6-11)/VIP-(7-28) hybrid, i.e., KPRRPYTDNYTRLRKQXMAVKKYLNSILN (SEQ ID NO:7), wherein X is norleucine;
[Acetyl-His¹, D-Phe², Lys¹⁵, Arg¹⁶, Leu¹⁷] VIP (1-7)/GRF (8-27) hybrid with C-terminal amidation, i.e., PG 97-269 or Ac-HEDAVFTNSYRKVLKRLSARKLLQDIL* (SEQ ID NO:8), a hybrid peptide of VIP residues 1-7 (HFDAVFT; SEQ ID NO:9) and GRF residues 8-27 (NSYRKVLKRLSARKLLQDIL; SEQ ID NO:10);
[Acetyl-His¹, D-Phe²] R2P3 with C-terminal amidation, i.e., Ac-HEDAVFTNSYRKVLKRLSARKLLODILC* (SEQ ID NO:11);
[Acetyl-His¹, D-Phe²] R2P11 with C-terminal amidation, i.e., Ac-HfDAVFTNSYRKVLKRLSVRKLLODILC* (SEQ ID NO:12); and
[Acetyl-His¹, D-Phe²] R2P16 with C-terminal amidation, i.e., Ac-HfDAVFTNSYRKVLKRLSARKLLQSIL* (SEQ ID NO:13).

VIPR1 peptide antagonists as provided by the invention can be advantageously synthesized by any of the chemical synthesis techniques known in the art, particularly solid-phase synthesis techniques, for example, using commercially available automated peptide synthesizers. The VIPR1 peptide antagonists can be synthesized by solid phase or solution phase methods conventionally used for the synthesis of peptides (see, e.g., Merrifield (1963) J. Amer. Chem. Soc. 85:2149-54; Carpino (1973) Ace. Chem. Res. 6:191-98; Kent (1988) Ann. Rev. Biochem. 57:957-89; Gregg et al. (1990) Int. J. Peptide Protein Res. 55:161-214).

It is contemplated that the VIPR1 peptide antagonists disclosed herein may be modified with hydrocarbon or polyethylene glycol groups in order to provide improve properties such as solubility, bioavailability, and/or biological degradation.

The VIPR1 antagonists may also be prepared by molecular imprinting, i.e., via de novo construction of macro molecular structures, such as peptides, which bind to a particular molecule. See for example, Mosbach, (1994) Trends in Biochem. Sci. 19(9). Binding peptides, such as antibodies, may easily be prepared by generating antibodies to VIPR1 or by screening libraries to identify peptides or other compounds which bind to VIPR1.

In another aspect, the expression of VIPR1 is inhibited with a VIPR1 inhibitory RNA molecule. The VIPR1 inhibitory RNA molecule may function by preventing the transcription of the VIPR1 gene or preventing the processing or translation of VIPR1 mRNA when administered in vivo or in vitro to an interneuron which is otherwise competent to express active VIPR1 protein. Thus, for example, VIPR1 inhibitory RNA molecules include repressors which prevent induction and/or transcription of the VIPR1 gene and antisense molecules that selectively bind to VIPR1 DNA or RNA sequences and prevent the transcription or translation of the VIPR1 gene. Examples of suitable inhibitory RNA molecules include, but are not limited to, antisense molecules, ribozymes or double stranded RNA (dsRNA), RNA interference (RNAi), miRNA (e.g., miR-525-5p), and/or small interfering RNA (siRNA). For example, an antisense molecule can be an oligonucleotide of between 5-100, preferably 10-50, preferably about 30 nucleotides in length, and complementary to a portion of the mRNA sequence (including the coding sequence) of the VIPR1 gene. Ideally, antisense molecule is an oligonucleotide which hybridizes under physiological conditions to DNA comprising the VIPR1 gene or to an RNA transcript of the VIPR1 gene and, thereby, inhibits the transcription of the VIPR1 gene and/or the translation of the mRNA.

Another type of VIPR1 inhibitory RNA molecule is an siRNA which functions by RNA interference (RNAi). An siRNA molecule is preferably a double-stranded RNA molecule with a 19 base pair double-stranded region (corresponding to a sequence on the target gene or mRNA) with a 2-base overhang at both ends (preferably a TT dimer overhang at each end). The methods for design of the RNA's that mediate RNAi and the methods for transfection of the RNAs into cells and animals is well known in the art and are readily commercially available (Verma et al. (2004) J. Clin. Pharm. Ther. 28(5):395-404; Mello et al. (2004) Nature 431(7006)338-42; Dharmacon Research (Lafayette, CO); Pierce Chemical (Rockford, IL); Glen Research (Sterling, VA); Origene (Rockville, MD); Santa Cruz Biotechnology (Dallas, TX), and ChemGenes (Ashland, MA). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein.

Preferably, the VIPR1 inhibitor is an isolated molecule. An isolated molecule is molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid or peptide. Because an isolated molecular species of the invention may be admixed with a pharmaceutically acceptable carrier in a pharmaceutical preparation, the molecular species may include only a small percentage by weight of the preparation. The molecular species is nonetheless substantially pure in that it has been substantially separated from the substances with which it may be associated in living systems.

VIPR1 inhibitors used in the methods of this invention can be provided in a pharmaceutical composition including the VIPR1 inhibitor in admixture with a pharmaceutically acceptable carrier. The pharmaceutically acceptable carrier preferably is non-pyrogenic and may include, but is not limited to, saline, buffered saline, dextrose, and water. A variety carriers may be employed, e.g., 0.4% saline, 0.3% glycine, and the like. These solutions are sterile and generally free of particulate matter. These solutions may be sterilized by conventional, well-known sterilization techniques (e.g., filtration).

The pharmaceutical compositions may contain pharmaceutically acceptable auxiliary substances as required. Acceptable auxiliary substances preferably are nontoxic to recipients at the dosages and concentrations employed. Auxiliary substances may be used to maintain or preserve, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCL, citrates, phosphates, or other organic acids), bulking agents (such as mannitol or glycine), chelating agents (such as ethylenediamine tetra acetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring agents, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics, PEG, sorbitan esters, polysorbates such as polysorbate 20 or polysorbate 80, tromethamine, lecithin, or cholesterol), stability enhancing agents (such as sucrose or sorbitol), tonicity enhancing agents (such as alkali metal halides), delivery vehicles, diluents, excipients and/or pharmaceutical adjuvants. See Remington's Pharmaceutical Sciences (18th Ed., A. R. Gennaro, ed., Mack Publishing Company 1990).

Yet another preparation can involve the formulation of VIPR1 inhibitor in an injectable microsphere, bio-erodible particle, polymeric compound (such as polylactic acid or polyglycolic acid), bead, or liposome, that provides for the controlled or sustained release of the product which may then be delivered via a depot injection. Other suitable means for the introduction of the desired inhibitor include implantable drug delivery devices.

[A pharmaceutical composition to be used for in vivo administration typically must be sterile. This may be accomplished by filtration through sterile filtration the composition is lyophilized, membranes. Where sterilization using this method may be conducted either prior to, or following, lyophilization and reconstitution.

The concentration of the VIPR1 inhibitor of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected. If desired, more than one type of inhibitor, for example with different K_{d} for VIPR1 binding, can be included in a pharmaceutical composition.

Pharmaceutical compositions of the invention can be administered by any number of routes as described herein including, but not limited to, intravenous, intramuscular, intra-arterial, intramedullary, intrathecal, intraventricular, intracerebroventricular, intrathecal-lumbar, intracisternal, transdermal, subcutaneous, intraperitoneal, intranasal, parenteral, topical, sublingual, or rectal means.

Subjects suitable for treatment in accordance with the methods of this invention include those wanting to improve pitch-discrimination acuity, e.g., musicians and linguists, as well as those with amusia (tone deafness). Subjects of this invention are preferably human. Subjects may receive treatment with a VIPR1 inhibitor for several days, weeks or months depending on the level of pitch-discrimination acuity improvement desired and/or rate of improvement.

The following non-limiting examples are provided to further illustrate the present invention.

### Example 1: Materials and Methods

Animals. Mice (6-12 weeks old) of both sexes were used for the experiments. Generation of CD^{+/-}, PD^{+/-}, and DD^{+/-} murine models of WBS (Li et al. (2009) EMBO Mol. Med. 1:50-65; Segura-Puimedon et al. (2014) Hum. Mol. Genet. 23:6481-6494) and Gtf2ird1^{30 /-} mice (Young et al. (2008) Genes Brain Behav. 7:224-234) have been described previously. Gad2^{Cre}, PV^{Cre}, Ai14, Ai93, CaMKII^{ttA}, and Scnn1a^{Cre} mice were purchased from the Jackson Laboratory (JAX, Bar Harbor, ME). CD^{+/-}, PD^{+/-}, and DD^{+/-} mice were backcrossed with C57BL/6J mice for 5-10 generations. The care and use of animals were reviewed and approved by the Institutional Animal Care and Use Committee at St. Jude Children's Research Hospital.

Generation of Vipr1-Conditional Knockout (cKO) Mice. The Vipr1-cKO mouse model was created using CRISPR-Cas9 technology and direct embryo injection. Briefly, chemically modified sgRNAs (Synthego, Menlo Park, CA) were tested prior to embryo injection for activity in mouse Neuro2a cells stably expressing Cas9 and assayed by targeted next-generation sequencing (NGS) as previously described (Segura-Puimedon et al. (2014) Hum. Mol. Genet. 23:6481-6494). Resulting NGS data were analyzed using CRIS.py (Connelly et al. (2019) Sci. Rep. 9:4194). For animal model generation, ten 3- to 4-week-old C57BL/6J female mice from JAX were superovulated with 5 units of pregnant mare's serum gonadotropin (PMSG; ProSpec, East Brunswick NJ) and 48 hours later, with 5 units of human chorionic gonadotrophin (hCG; Sigma-Aldrich, St. Louis, MO). After overnight mating with C57BL/6J males, the females were euthanized, and oocytes were harvested from the ampullae. The protective cumulus cells were removed using hyaluronidase, and the oocytes were washed and graded for fertilization by observing the presence of two pronuclei. A mixture of the sgRNAs, Cas9, and Coralville, I ssODNs (single-stranded oligodeoxyribonucleotide) composed of 60 ng/ml of Cas9 protein (St. Jude Protein Production Core), 20 ng/ml of each sgRNA, and 5-10 ng/ml of each SSODN (Integrated DNA Technologies, Inc., Coralville, IA) were injected into the pronucleus of oocytes. The injected oocytes were then returned to culture media (M16 or Advanced-KSOM, both from Millipore, Burlington, MA) and later the same day transferred to Day 0.5 pseudo-pregnant fosters. Pups were born after 19 days gestation and sampled at Days 7-10 for genotyping via targeted NGS. Animals positive for both LoxP-site integration events were weaned at Day 21. At 6 weeks of age, they were backcrossed to C57BL/6J mice and then bred to homozygosity. Editing construct sequences and relevant primers are listed in Table 1.

TABLE 1

| | | SEQ ID |
|---|---|---|
| Name | Sequence (5′ to 3′) | NO: |
| CAGE289.Vipr1.g3 | AAGUGGGAUAAGAGUUCAUC | 14 |
| spacer | | |
| CAGE289.g3.sense.ss | *GGTTTTTGTAGGGGACAATTTAGAAGTGG | 15 |
| ODN | GATAAGAGTTCATAACTTCGTATAATGTAT | |
| *AltR modifications | GCTATACGAAGTTATGGATCCATCTGGGCC | |
| | TAGGATGGGTTATAGCCTGGGTTGGGGTTGG | |
| CAGE289.DS.F | GGAGCCAAGAGTCCTGAGAAGGCCC | 16 |
| CAGE289.DS.R | CACAGGCTTTCGGAGTAGGGGGCCA | 17 |
| CAGE290.Vipr1.g10 | AGCCACAGCUAGACCCUUAA | 18 |
| spacer | | |
| CAGE290.DS.F | CCCTCACGTCACGAGCCCAGTCCAA | 19 |
| CAGE290.DS.R | TTTGTGCTGATGGGCTGCTGCAGGG | 20 |
| CAGE290.g10.anti.ss | *CCTCCCTCCTTGGGTAGCCCAGCAGCCAC | 21 |
| ODN | AGCTAGACCCTATAACTTCGTATAGCATAC | |
| *AltR modifications | ATTATACGAAGTTATGGATCCTAAAGGTAG | |
| | TTTCCAGATAAGAGCTGGGAACTCCCCAGA | |
| | T | |

Generation of Vipr1-OE Transgenic Animal. For the generation of the Vipr1-OE vector, full-length mVipr1 cDNA was subcloned into the multiple cloning site of a pCAGGs-LSL-IRES-EGFP backbone by using the following primer sets: mVipr1 F (5′-TAGTGGATCCCCCGGATGCGCCCTCCGAGC-3′; SEQ ID NO:22) and mVipr1 R (5′-CGAGGTTAACGAATTTCAGACCAGGGAGACCTCCGC-3′; SEQ ID NO:23) and linearized with restriction enzyme PvuI for pronuclear microinjection. Female C57BL/6J mice (3-4 weeks) were superovulated with gonadotrophin injections 1 and 2 days prior to the experiment; the first 5 units of gonadotrophin isolated from PMSP, then 48 hours later, 5 units of hCG. Females were then mated to C57BL/6J males. Fertilized zygotes were collected the following morning in M2 or Advanced-KSOM media, and cumulus cells were stripped from the zygotes with hyaluronidase. The cytoplasm of each zygote was microinjected with 1-5 ng/μL of linearized pCAG-LSL-Vipr1-IRES-eGFP DNA diluted in IDTE (a Tris-EDTA buffer at pH 7.5). After being maintained in culture in M16 or Advanced-KSOM media, the injected zygotes were transferred into the oviducts of pseudo-pregnant females. At 7-10 days of age, pups were sampled for genotyping and fluorescence in situ hybridization (FISH) confirmation of the genomic insertion of the pCAG-LSL-Vipr1-IRES-eGFP transgene. FISH was performed as follows: purified pCAG-LSL-Vipr1-IRES-eGFP DNA was labeled by nick translation using a red dUTP (ALEXA FLUOR® 594-5-dUTP, Molecular Probes, Eugene, OR), and control probes were labeled with a green dUTP (ALEXA FLUOR® 488-5-dUTP, Molecular Probes, Eugene, OR). Mouse lung fibroblast from transgenic mice were grown in culture and harvested by conventional cytogenetic methods as a source of metaphase chromosomes. The labeled transgene probe was first hybridized to transgenic metaphases to identify the site of insertion. A second hybridization using the transgene probe and a chromosome-specific control probe was performed to confirm the identity of the chromosome bearing the transgene insertion. Hybridizations were carried out using a hybridization buffer containing 50% formamide, 10% dextran, and 2×saline-sodium citrate buffer (SSC). Fixed slides were denatured in 70% formamide 2×SSC, at 80° C. Post-hybridization washes were done using 50% formamide 2×SSC at 37° C. Slides were mounted in VECTASHIELD® mounting medium containing DAPI, and images were acquired using a NIKON® Eclipse 80i with a X100, 1.40-NA Plan Apo objective and CytoVision version 7.7 (Leica Biosystems).

Mouse Behavioral Tests: Frequency Discrimination (Auditory) Acuity Test. Pitch-discrimination acuity was assessed using pre-pulse inhibition (PPI) of the acoustic startle response via a hardware-computer interface (Kinder Scientific, Chula Vista, CA), according to established methods (Aizenberg et al. (2015) PLOS Biol. 13:e1002308; Blundon et al. (2017) Science 356:1352-1356). In brief, a background pure tone (16.4 kHz) was played at a sound pressure level (SPL) of 70 dB throughout the session, unless otherwise noted. Each session was split into four blocks. Block 1 included a 5-minute acclimation period in which the background tone was played. Block 2 included nine startle trials in which a 120-dB SPL, 20-ms white noise (WN) burst was played. Block 3 included prepulse trials and 10 startle-only trials in a pseudo-random order. Each pre-pulse trial included a 70-dB SPL 80-ms pre-pulse (pure-tone frequency was 0%, 1%, 2%, 4%, 8%, 16%, or 32% lower than that of the background tone), followed by a 120-dB SPL, 20-ms WN startle pulse, and then returned to the background tone after the startle. Every trial in Block 3 was presented 10 times. Block 4 included three startle trials to identify any habituation over the session. The intertrial interval was 10-20 seconds, and the startle magnitude was the maximum force exerted immediately after the startle pulse. For all trials,. wav files were created using Audacity 2.1.2 (Audacity, open source). PPI percentage was calculated from Block 3 data as follows: [1−(pre-pulse trial/average startle only trial)]*100. Values in Block 2 trials were compared with those in Block 4 as an internal control for startle attenuation over the course of the session. Each animal then had a 3-parameter logistic regression curve fitted to the PPI percentages at each pre-pulse frequency to determine the frequency at which 50% of the total acoustic startle inhibition was achieved, subsequently called the frequency-discrimination threshold (FDT); animals with an r²<0.7 were excluded from further analyses. FDT values were then analyzed using a t-test, a one-way ANOVA, or a paired t-test, as appropriate. Pure tone frequencies and sound intensities were calibrated daily by using the sound level meters NL-52 (Rion Co., Ltd) and SMSPL Rev B (Kinder Scientific), respectively. In chemogenetic experiments administered on consecutive days, animals were intraperitoneally (i.p.) injected with designer receptor exclusively activated by designer drug (DREADD) agonist C21 (1 mg/kg in 0.9% saline; Tocris, Minneapolis, MN) or vehicle 30 minutes before undergoing the PPI test, in a counterbalanced manner to avoid order-of-treatment effects.

Mouse Behavioral Tests: Auditory Brainstem Response Test. Auditory brainstem response (ABR) experiments were performed as previously described (Chun et al. (2017) Nat. Med. 23:39-48; Mellado Lagarde et al. (2014) Proc. Natl. Acad. Sci. USA 111:16919-16924). Briefly, mice were anesthetized with Avertin (0.6 mg/g bodyweight, i.p.), and ABR was measured using a Tucker Davis Technology (TDT) System III with RZ6 Multiprocessor and BioSigRZ software. Sounds were delivered via the MF-1 speaker in the open-field configuration. ABR waveforms were recorded using subdermal needles placed at the vertex of the skull, below the pinna of the ear, and at the base of the tail. The needles were connected to a low-impedance head stage (TDT, Alachua, FL) and fed into the RZ6 multiprocessor through a preamplifier (RA4PA, Gain 20X, TDT, Alachua, FL). ABR waveforms were averaged from 500 presentations of a tone (21 tones/second) in the alternating phase and were band-pass filtered (300 Hz-3 kHz). The ABR threshold was defined as the minimum sound intensity that elicited a wave above the noise level. All ABR experiments were conducted in a sound booth (Industrial Acoustic Company (IAC), Hampshire, UK; Model 120A double wall).

Single-Cell Electrophysiology: Auditory Thalamocortical Brain Slices. Acute primary thalamocortical (TC) slices (400-μm thick) containing the left ACx and the left ventral part of the medial geniculate nuclei (MGv) of the thalamus were prepared as previously described (Chun et al. (2014) Science 344:1178-1182; Cruikshank et al. (2002) J. Neurophysiol. 87:361-384). Briefly, mouse brains were removed and placed in cold (4° C.) dissecting medium containing 125 mM choline-Cl, 2.5 mM KCl, 0.4 mM CaCl₂, 6 mM MgCl₂, 1.25 mM NaH₂PO₄, 26 mM NaHCO₃, and 20 mM glucose (300-310 mosm), equilibrated with 95% O₂/5% CO₂. TC slices were obtained from the left hemisphere using a slicing angle of 15° to horizontal. Slices were transferred to artificial cerebral spinal fluid (ACSF) containing 125 mM NaCl, 2.5 mM KCl, 2 mM CaCl₂, 2 mM MgCl₂, 1.25 mM NaH₂PO₄, 26 mM NaHCO₃, 20 mM glucose (300-310 mOsm), equilibrated with 95% O₂/5% CO₂ at 34° C. for 30 minutes followed by 1-hour at room temperature prior to use. Slices were transferred to a recording chamber mounted on an upright microscope (Olympus) and superfused (˜2 mL/min) with warm (30-32° C.) ACSF. Slices were viewed with a CCD camera using infrared differential interference contrast (IR-DIC) optics. Thalamorecipient pyramidal neurons in L4 (˜300 μm from the pia) were identified by soma shape and size and by a large visible apical dendrite projecting toward the pia. If recorded in current-clamp mode, pyramidal neurons were additionally verified as regularly spiking. Fast-spiking (FS) interneurons were identified as having nonpyramidal shape and multipolar dendritic projections from the soma. The FS phenotype was verified by recording in current-clamp mode. Mice with tdTomato genetically expressed in PV⁺ cells were used in a subset of experiments in which fluorescently labelled soma were targeted using the microscope's epifluorescence.

Single-Cell Electrophysiology: Whole-Cell Recording. Whole-cell recordings were made with patch pipettes (3-5 MOhm) using a Multiclamp 700B amplifier, digitized (10 kHz) with a Digidata 1440, and recorded using pCLAMP 10 software (all Molecular Devices, San Jose, CA). In all experiments, membrane potentials were corrected for a liquid junction potential of −10 mV. In voltage-clamp recordings, series resistance, input resistance, and holding current were monitored for stability. During current-clamp recordings, pipette capacitance and series resistance were compensated using the amplifier's circuits. Input resistance and membrane resting voltage were monitored during recordings. Cells with series resistance over 40 MOhms in voltage-clamp recordings and 30 MOhms in current-clamp recordings or cells that changed resistance values more than 20% over duration of recordings were rejected. Drugs were added to ACSF or locally applied via continuous pressure ejection from a large-diameter pipette placed in the slice near the recorded cell. Pressure ejection of control ACSF caused no detectable effect on neurons.

For standard voltage-clamp recordings, patch pipettes were filled with an internal solution containing 125 mM CsMeSO₃, 2 mM CsCl, 10 mM HEPES, 0.1 mM EGTA, 4 mM ATP-Mg₂, 0.3 mM GTP-Na, 10 mM creatine phosphate-Na₂, 5 mM QX-314 (a quaternary ammonium lidocaine derivative), and 5 mM TEA-Cl (tetraethylammonium; pH 7.4, 290-295 mOsm). For current-clamp recordings, internal solution contained 115 mM potassium gluconate, 20 mM KCl, 10 mM HEPES, 4 mM MgCl₂, 0.1 mM EGTA, 4 mM ATP-Mg₂, 0.4 mM GTP-Na, and 10 mM creatine phosphate-Na₂ (pH 7.4, 290-295 mOsm). For voltage-clamp recording of voltage-gated Ca²⁺ currents, external CaCl₂ was replaced with 3 mM BaCl₂, 0.5 M tetrodotoxin (TTX) included in the ACSF, and EGTA and QX-314 were omitted from the internal solution. For voltage clamp I-V curves, cells were hyperpolarized to −90 mV followed by steps of increasing depolarization amplitude (duration as indicated). Current intensity was corrected for linear leak current, as determined from a brief −5-mV step from rest. Na⁺ and Ca²⁺ current density was quantified as the peak inward current divided by the membrane capacitance. K⁺ current density was quantified as the steady-state outward current divided by the membrane capacitance. The Iₕ density was determined by delivering 2-second hyperpolarizing pulses from rest and measuring the inward current “sag” divided by the membrane capacitance. To determine the Ca²⁺ current threshold, 1-second ramps from −90 to +30 mV were delivered. Responses were leak-subtracted, and the threshold was quantified as the peak of the second derivative of the current signal.

Spontaneous synaptic inputs were recorded with neurons held at −70 mV for excitatory postsynaptic potentials (with or without inhibitory inputs blocked by 100 μM picrotoxin (PTX), as indicated) and 0 mV for inhibitory synaptic inputs (with or without excitatory inputs blocked with 3 mM kynurenic acid as indicated). For miniature synaptic events, 0.5 μM TTX was included in the ACSF. Spontaneous activity was recorded for 5-10 minutes beginning at least 2 minutes after whole-cell break-in. Spontaneous excitatory synaptic currents (sEPSCs) were automatically detected using miniAnalysis (Synaptosoft) as deviations of more than 5× the baseline root mean squared noise level.

Current clamp input-output curves were obtained by delivering 1-second current pulses of increasing amplitude. Rheobase was determined by delivering a current ramp at 300 pA/s and measuring the current intensity that elicited the first spike. Input resistance was calculated either from a small hyperpolarizing test pulse or from the slope of the initial, linear response to the ramp. Individual spike properties (threshold, after-hyperpolarization potential [AHP], half-width, etc.) were measured using MiniAnalysis (Synaptosoft). Threshold was determined as the peak of the second differential of the voltage signal. AHP was determined as the negative peak voltage relative to the threshold.

To generate TC input-output curves, TC pluripotent stem cells (PSCs) were evoked by current pulses (intensity 0.1-1 mA, duration, 100 us) delivered to the thalamic radiation via tungsten concentric bipolar electrodes (FHC) using a stimulus isolator (Iso-flex; A.M. P. I.). Monosynaptic EPSC amplitude was quantified as the initial slope of the inward current response.

In Vivo Viral Injections: Generation of pAAV-hDLX-Vipr1-T2A-eGFP and pAAV-hDLX-Vipr1-T2A-tdTomato Plasmids. Coding sequences of the mVipr1 (GENBANK Accession number: NM_011703.4) were amplified with primers, Vipr1 F (5′-CTTAAGAAAGGTCGACCACCATGCGCCCTCCGAGCCT-3′; SEQ ID NO:24) and Vipr1 R (5′-TGCCCTCTCCGGATCCGACCAGGGAGACCTCCGC-3′; SEQ ID NO:25) from cDNA, generated from reverse-transcribed mouse whole-brain RNA using the SUPERSCRIPT@ First-Strand Synthesis RT-PCR Kit (Invitrogen, Carlsbad, CA), inserted into pAAV-hDLX-T2A-eGFP vector plasmid (modified from Addgene plasmid 83895) by infusion cloning (Takara Bio Inc., San Jose, CA).

In Vivo Viral Injections: Generation of pAAV-hDlx-Vipr1-T2A-TdTomato Plasmid. The protein-coding sequence of tdTomato was PCR-amplified from pGP-AAV-CAG-FLEX-jGCaMP7s-WPRE (Addgene) by using two PCR primers, tdTomato F (5′-CTTAAGAAAGGTCGACCACCATGGTGAGCAAGGGCGAG-3′; SEQ ID NO:26) and tdTomato R (5′-CCGCTATCACAGATCACTAGTCTTGTACAGCTCGTCC-3′; SEQ ID NO:27) and replaced the eGFP-coding sequence of pAAV-hDLX-VIPR1-T2A-EGFP by infusion cloning. The pAAV-hDlx-Flex-GFP-Fishell_6 plasmid is known (Addgene, Watertown, MA).

Viral Injections: Surgery. Mice were In Vivo anesthetized with 2% isoflurane (in pure oxygen), and under aseptic conditions, a midline incision was made in the scalp. Virus was injected bilaterally into the primary ACx (250 nL per site at a rate of 30 nL/min; coordinates: 2.2 mm caudal to bregma, 0.3 mm medial to the dorsal insertion of the temporalis muscle onto the skull, and injection depth 0.8 mm).

In Vivo Two-Photon Calcium Imaging. Two-photon calcium imaging was used in GCaMP6f^{E×N-L4}; WT and GCaMP6f^{E×N-L4}; CD^{+/-} mice as previously described (Blundon et al. (2017) Science 356:1352-1356). Mice were anesthetized with a mixture of ketamine/xylazine (100/10 mg/kg body weight) and subsequent injections of 50 mg/kg ketamine. Under aseptic conditions, a 2- to 3-g stainless steel headpost was fixed to three miniature screws in the skull and cemented into place with dental cement. Using the headpost to secure the animal's head, the lateral temporalis muscle was removed to reveal the skull overlying the ACx. A craniotomy was made using a 1.5-mm biopsy punch, and a plastic well was cemented around the craniotomy to hold saline. The overlying dura was carefully removed, and a 3-mm glass coverslip was cemented over the cranial window. To reduce postoperative pain, decrease inflammation, and eliminate infection, each mouse was given subcutaneous injections of meloxicam (2 mg/kg), Baytril (5 mg/kg), and dexamethasone (2 mg/kg), and amoxicillin (0.3 mg/mL) in the drinking water. The animals received this postoperative care for the duration of the experiments.

After recovery, mice were acclimated to the head-fixed setup. For at least 3 days prior to imaging, the animal was stabilized on a rotating disc under the two-photon microscope while the head was secured in place with the headpost. Acclimation began with 15-minute intervals and progressed to 1-hour intervals. During acclimation and imaging, animals were in the dark, surrounded by a sound-attenuating chamber. To determine differences in spontaneous firing patterns between genotypes and sound-evoked firing patterns, animals were imaged during 30 minutes of silence and 30 minutes of sound delivery. GCaMP6f fluorescence in L4 neurons located 300-400 μm beneath the pial surface was monitored with the Olympus multiphoton imaging system (FluoView FV1000) and an Insight tunable femtosecond-pulsed laser unit (Spectra-Physics, Lane Milpitas, CA). Neurons expressing GCaMP6f were imaged with a 25× water immersion objective (Olympus XPlan N) using an excitation wavelength of 930 nm with a resonant scanner at a rate of 10 frames/second with a field of view of 512 μm×512 μm. Tones were generated with OpenEx software and an RZ6 signal processor with 100-MHz processing speed (TDT) and delivered through a free-field electrostatic speaker placed 10 cm from the contralateral ear of the animal. During sound-delivery experiments, the sound-stimulation software triggered the start of the microscope-scanning software. GCaMP6f fluorescence was measured in response to pure tones with frequencies ranging from 4.8 to 29.4 kHz, with intensities of c 10-70 dB SPL (60-0 dB attenuation, respectively), and duration of 50 ms played at 1 Hz in pseudo-random order. Cells were included in the analysis if they were in focus during both the sound and silent conditions.

Video Processing. Videos were corrected for large movement artifacts with a custom Matlab routine. To motion stabilize the videos, each frame was aligned to a stable reference frame using a nonrigid image-registration algorithm, as previously described (Blundon et al. (2017) Science 356:1352-1356; Rueckert et al. (1999) IEEE Trans. Med. Imaging 18:712-721). Following stabilization, video segments with high movement artifacts were pruned from the video sequence using a custom Matlab code.

Prior to automatic cell identification, ground truths for cells and delta F/F (DF/F) peaks were established independently by two investigators. Next, the background was subtracted, and videos were down-sampled and segmented. A particle mask analysis was applied to create a region of interest (ROI) surrounding each soma based on the standard deviation of the Z-projection of fluorescence for each video sequence. Fluorescence signals were normalized to the baseline, and DF/F of the peak amplitudes was calculated as the change in fluorescence over baseline fluorescence levels×100%.

Calcium Imaging and Data Processing. The image-processing pipeline used a custom Fiji (Schindelin et al. (2012) Nat. Methods 2012 97 9:676-682) macro to automatically quantify raw calcium traces from the time-lapse images. A custom R package was then used to automatically identify individual cells and deconvolve the traces. Both custom R and python scripts were used to calculate the metrics used. The DF/F image was calculated by using the following equation:

$$\frac{DF}{F} = \frac{F - {F}_{mean}}{{F}_{mean}} ,$$

where F was the raw calcium signal image, and Fmean was the corresponding temporal moving mean filter with a 10-second window.

Cell Body Segmentation. The Ilastik software package (Berg et al. (2019) Nat. Methods 16(12):1226-1232) was used to train a classifier to segment all cell bodies, frame by frame, that had calcium intensities above the background level. A background-subtracted image was used as an input for the segmentation. A temporal moving median filter with a 10-second window was used to remove the background intensity for all pixels in the 512×512 time-lapse image. The ROIs defined by the segmented cell bodies in each frame were used to calculate the mean intensity of the calcium signal. The mean intensity and the frame number, location, and area of the ROIs of a time-lapse recording were stored in comma-separated value (CSV) files.

Individual Cell Identification. The XY coordinates of the cell bodies detected in the entire recording of a cell were typically clustered within a few pixels. The spread of the coordinates of the centers depended on the degree of motion artifact. Hierarchical clustering of the XY coordinates was used to identify individual cells. The hierarchical tree-cut height parameter was adjusted to minimize over-segmentation (multiple cell IDs assigned to one biological cell) and under-segmentation (single-cell ID assigned to multiple biological cells).

Deconvolution. The OASIS software package (Friedrich et al. (2017) PLoS Comput. Biol. 13:e1005423) was used to deconvolve raw calcium signal traces. A rise time of 45 ms was used and 142 ms as the decay time parameters (Chen et al. (2013) Nature 499:295-300). All the local maxima of the deconvolved trace above the threshold were used as cell-firing events.

Decoder. To analyze the differences in sound discriminability based on L4 excitatory activity, a linear frequency and cue decoder was constructed based on the deconvolved calcium traces, inspired by the linear decoder (Kingsbury et al. (2020) Neuron 107:941-953.e7). Deconvolved traces were z-scored. To reduce the dimensionality of the training data, principal component analysis (PCA) was performed, and the top 20 principal components (PCs) were extracted. A logistic-regression model that includes the projections onto the PCs as input was used. For the frequency decoder, the model was trained to predict the frequency of the cue that was most recently presented. Unless otherwise noted, this was trained on and applied to only the cue frame and the four frames after the cue. The sound-nosound decoder was trained to predict whether a frame was a cue frame or not. For this decoder, only cue frames and the five frames preceding a cue frame were used. Five-fold cross validation was used to estimate the decoder performance as follows: data were divided into five blocks and from these blocks, five train/validation splits were constructed, where each split used one block for validation and the remaining four blocks for training. Mean validation accuracies over all splits were reported in the text. To avoid leakage, splits were contiguous periods of the entire recording and were performed such that no individual cue-presentation period was split between train and validation.

To determine what factors most contributed to the significant difference in frequency discrimination between wild-type (WT) the same and WBS, decoder analysis was performed using modifications or subsets of the original data described above. First, to investigate if the difference in discriminability was the result of different numbers of cells being reliably imaged and analyzed in WBS vs WT, the data sets were balanced such that the input to the decoder for all recordings was a set of 50 randomly selected cells of the entire population. Second, to investigate if the difference in discriminability was a result of different proportions of sound-responsive cells in WBS vs WT, these proportions were matched in the following way: a cell was considered sound responsive if its mean deconvolved signal at cue frames was more than 1.96 standard errors above its mean baseline level (mean activity over the five pre-cue frames). For each recording, cells were randomly selected, such that 20% of them were sound-responsive. Finally, to investigate if the difference in discriminability was a result of the choice of frames over which the decoding was performed, the frequency-decoding analysis was repeated only using the cue frame and the frame immediately after it. This decoder analysis was performed with custom code in python using the scikit-learn package (Pedregosa et al. (2011) J. Mach. Learn. Res. 12:2825-2830).

Isolation of GAD⁺ Interneurons from the ACx. Gad2^{Cre}; Ai14; WT or Gad2^{Cre}; Ai14; CD^{+/-} mice were euthanized via cervical dislocation and decapitated. The ACx was isolated and washed with cold Earle's Balanced Salt Solution

(Worthington Biochemical Company, Lakewood, NJ) and then placed in plain neurobasal medium (Thermo Fisher Scientific). The tissue was dissociated with activated papain (Worthington Biochemical Company) and DNAse I (Sigma-Aldrich) for 30 minutes at 37° C. Then it was triturated by repeated gentle pipetting with a 2-mL glass pipette. Tissue digestion was stopped by adding reconstituted BSA-ovalbumin solution (Worthington Biochemical Company). The resulting single-cell suspension was filtered through a 40-μm cell strainer (BD Biosciences, San Jose, CA), centrifuged at 300×g for 5 minutes at room temperature, washed once, and resuspended with cold Earle's Balanced Salt Solution. The single-cell suspension was then FACS-sorted by an Aria Fusion cytometer (BD Biosciences) equipped with blue (488 nm), yellow/green (561 nm), red (640 nm), and violet (405 nm) lasers to isolate tdTomato⁺ cells. A 100-μm nozzle was used for sorting and BD FACS Diva Software (BD Biosciences) was used for data acquisition and analysis.

Stranded Total RNA-seq. Total RNA was isolated from brain tissue or organoids by using mirVana™ RNA isolation kit (Life Technologies, Carlsbad, CA), quantified using the Quant-iT™ RIBOGREEN® RNA assay (Thermo Fisher Scientific), and quality checked by the 2100 Bioanalyzer RNA 6000 Nano assay (Agilent Technologies, Inc., Santa Clara, CA) or 4200 TapeStation High Sensitivity RNA ScreenTape assay (Agilent Technologies, Inc.) prior to library generation. Libraries were prepared from total RNA with the TRUSEQ® Stranded Total RNA Library Kit Prep according to the manufacturer's instructions (Illumina, San Diego, CA). Libraries were analyzed for insert-size distribution using the 2100 BioAnalyzer High Sensitivity kit (Agilent Technologies, Inc.), 4200 TapeStation D1000 ScreenTape assay (Agilent Technologies, Inc.), or 5300 Fragment Analyzer NGS fragment kit (Agilent Technologies, Inc.). Libraries were quantified using the Quant-iT™ PICOGREEN® dsDNA assay (Thermo Fisher Scientific) or by low-pass sequencing with a MISEQ® nano kit (Illumina). Paired-end 100-cycle sequencing was performed on a NOVASEQ® 6000 (Illumina).

RNA-seq Data Analysis. Total stranded RNA-seq data were processed by the internal AutoMapper pipeline. Briefly, the raw reads were first trimmed (Trim-Galore version 0.60), then mapped to the human genome assembly GRCh38 (STAR v2.7; Dobin et al. (2013) Bioinformatics 29:15-21). The gene-level values were then quantified (RSEM v1.31; Li & Dewey (2011) BMC Bioinforma. 12:1-16) based on GENCODE annotation (v31). Low-count genes were removed from the analysis by using a CPM cutoff corresponding to a count of 10 reads and only confidently annotated (levels 1 and 2 gene annotation), and protein-coding genes were used for differential-expression analysis. Normalization factors were generated using the TMM method (Robinson & Oshlack (2010) Genome Biol. 11:1-9), counts were normalized using voom (Law et al. (2014) Genome Biol. 2014 152 15:1-17); and normalized counts were analyzed using the ImFit and eBayes functions (R limma package version 3.42.2). The significantly up- and down-regulated genes were defined by at least two-fold changes and an adjusted p-value <0.05.

Quantitative RT-PCR. Total RNA was isolated from the tissue or cells by using the mirVana™ microRNA Isolation Kit (Life Technologies). The iScript kit (Bio-Rad Laboratories, Hercules, CA) was used to synthesize cDNA from the isolated total RNA and the quantitative RT-PCR was performed using SYBR® Green (Life Technologies). The following forward primers were used for analysis: mVipr1 F (5′-AGTATGGATGAGCAGCAACAGA-3′; SEQ ID NO:28), mVipr1 R (5′-AGATAGCCATGGCAACCAGG-3′; SEQ ID NO:29), mGtf2ird1 F (5′-ACTGTGACATCCCCACCAAC-3′; SEQ ID NO:30), mGtf2ird1 R (5′-GAGTCTAAGGCGGACACCAG-3′; SEQ ID NO:31), and GFP F (5′-CTACGGCAAGCTGACCCTGAAGTT-3′; SEQ ID NO:32), GFP R (5′-CTCGGCGCGGGTCTTGTAGTT-3′; SEQ ID NO:33). The following primers were used for loading controls: mU6 snRNA F (5′-CGCTTCGGCAGCACATATAC-3′; SEQ ID NO:34) and mU6 snRNA R (5′-TTCACGAATTTGCGTGTCAT-3′; SEQ ID NO:35). Expression levels of Vipr1, Gtf2ird1, or GFP were normalized to the housekeeping gene U6 for each sample. Samples from each mouse were run in triplicate.

Western Blot Analysis. Brain tissue was resuspended with RIPA buffer containing protease inhibitors and sonicated twice at 15% amplitude for 10 seconds in a Bronson sonifier on ice. Supernatant was collected from total-protein lysate by centrifugation at 13,000×g for 10 minutes at 4° C. After quantification of the supernatant fraction by BCA assay (Thermo Fisher Scientific), 10 mg of the protein sample was fractionated by using the SDS-polyacrylamide gel electrophoresis (SDS-PAGE) and transferred onto a PVDF membrane (Thermo Fisher Scientific). After incubation with 5% (wt/vol) nonfat dry milk in TBST (10 mM Tris. pH 8.0, 150 mM NaCl, and 0.5% (vol/vol) polysorbate 20) for 30 minutes, membranes were incubated with anti-VIPR1 (1:250 dilution; Thermo Fisher Scientific) or anti-ACTB (actin) (1:5,000 dilution; Sigma-Aldrich) antibodies at room temperature for 1 hour. Membranes were washed for 5 minutes three times and incubated with a 1:3000 dilution of horseradish peroxidase-conjugated anti-rabbit or anti-mouse antibodies (Santa Cruz Biotechnology, Dallas, TX) at room temperature for 1 hour. Blots were washed with TBST three times and developed by using the ECL system (Pierce Biotechnology Inc., Rockford, IL).

Histology and Immunohistochemistry. Mice were deeply anesthetized and intracardially perfused with 4% paraformaldehyde in 0.1 mol/L phosphate buffer (pH 7.4), and brains were fixed overnight. Each brain was sliced (50 μm) coronally with a Leica vibratome. The brain sections were preincubated in sodium citrate buffer (10 mM citrate buffer, pH 6.0) at 80° C. for 20 minutes, cooled to room temperature, and washed in 1× phosphate-buffered saline (PBS) for 20 minutes twice. Sections were incubated in PBS-blocking buffer (5% goat serum, 3% BSA, 0.2% TRITON™ X100, in PBS) for 1 hour at room temperature and incubated with the following primary antibodies: VIPR1 (1:250, Invitrogen), parvalbumin (PV) (1:5000, Swant), GFP (1:1000, Abcam), GABA (1:1000, Sigma Aldrich) for 48 hours at 4° C. Appropriate ALEXA FLUOR® dye-conjugated secondary antibodies (1:1000, Thermo Fisher Scientific) were used to detect primary antibody binding for 48 hours at 4° C. DAPI (Invitrogen) was used as the nuclear counterstain. Images of immunostained human sections were quantified using Fiji (ImageJ). PV-stained sections were thresholded and used to automatically generate ROIs around PV⁺ cell bodies. Average pixel intensity of VIPR1 staining of the same slices was quantified within those ROIs.

Generation of hIPSC Culture with Isogenic WBS Microdeletion. The NSUN5-GTF2IRD2 heterozygous microdeletion was introduced into TP-190a hiPSC clones using the CRISPR-Cas9 method. Three TP-190a hiPSC clones were obtained by reprogramming dental pulp stem cells from a healthy male (ALSTEM) using episomal plasmids. All three clones expressed pluripotency markers, had a normal karyotype (G-Banding and SNP Microarray), and displayed high neural differentiation potential. One clone (#2) was selected for differentiation experiments. TP190a hiPSCs were pretreated with STEMFLEX® culture medium (Thermo Fisher Scientific) supplemented with 1× REVITACELL® Supplement (Thermo Fisher Scientific Scientific) for 1 hour. Then, approximately 2×10⁶ cells were transiently co-transfected with precomplexed ribonuclear proteins composed of 150 pmol of each chemically modified sgRNA, 100 pmol SpCas9 protein (St. Jude Protein Production Core), and 500 ng pMaxGFP (Lonza). The transfection was performed via nucleofection (Lonza, 4D-NUCLEOFECTOR™ X-unit) using solution P3 and program CA-137 in a large (100 mL) cuvette according to the manufacturer's recommended protocol. At 12 days post-transfection, cells were sorted based on viability and plated onto Vitronectin XE (Stem Cell Technologies)-coated plates into prewarmed (37° C.) STEMFLEX® media supplemented with 1× CLONER® serum-free supplement (Stem Cell Technologies). Clones were expanded, screened, and verified for the desired deletion via Sanger sequencing. Zygosity was confirmed using 5′, 3′, and internal primers. Editing construct sequences and relevant primers are listed in Table 2. Of note, the CAGE865. GTF2IRD2. g3 sgRNA was designed to a unique sequence in the TP190a genome that differs from the reference genome.

TABLE 2

| | | SEQ ID |
|---|---|---|
| Name | Sequence (5′ to 3′) | NO: |
| CAGE636.NSUN5.g11 spacer | UUGAACGGGUCGAGGUGCCA | 36 |
| CAGE865. GTF2IRD2.g3 spacer | AAUGGCGGCGUCGGCGGCGU | 37 |
| CAGE865.GTF2IRD2.DS.Deletion.F2 | CCCCGAAGCGTGCTCGT | 38 |
| CAGE636.NSUN.DS.Deletion.R2 | GCGGCTCTTTGCTGTCTCTT | 39 |
| CAGE636.DS.internal.F | ACTGACCAGCACACCAACAA | 40 |
| CAGE636.DS.internal.R | GCTCAACGGTGGAAAGAGGA | 41 |
| CAGE636.DS.5′.F | GGGGCCGTTTCTCTTGCAGGC | 42 |
| | TAGC | |
| CAGE636.DS.5′.R | TCTTTCTCTTTGGGGCTGGGC | 43 |
| | TGGG | |
| CAGE865.DS.3′.F | AAAAAGGAGGGCGAGTGGCGA | 44 |
| | GCAG | |
| CAGE865.DS.3′.R | CCCCACCCTCACACCTCTGGT | 45 |
| | CCTG | |

The control hiPSC line TP-190a and the isogenic microdeletion line TP-190a-NSUN-GTF2IRD2-DEL clone 2F9 were maintained in culture on hES-qualified solubilized basement membrane sold under the tradename MATRIGEL® (Corning) in complete MTESR®1 medium (STEMCELL Technologies) at 5% O₂, 37° C., and 5% CO₂. The cultures were passaged with Versene (Thermo Fisher Scientific).

Cortical Organoids. Human cortical organoids were generated using a method adapted from a previously published protocol (Rai et al. (2021) Cell Metab. 33:1137-1154.e9). Briefly, hiPSC cultures were dissociated into single cells with ACCUTASE® (Innovative Cell Technologies) and plated into low-attachment 96-well V-bottom plates (Sbio) at 9000 cells/well, in EB (DMEM:F12, media 20% Knockout Serum Replacement (Life Technologies), 3% ES-FBS (Sigma), 1× Glutamax (Gibco), 1× β-mercaptoethanol (Gibco), 1× antibiotic-antimycotic (Gibco) supplemented with 5 mM SB-431542 (TGFB inhibitor, Tocris), 2 mM dorsomorphin (Tocris), 3 mM IWRle (Wnt inhibitor, EMD Millipore), 1% v/v growth factor-reduced solubilized basement membrane sold under the tradename MATRIGEL® (Corning), and 2 mM thiazovivin (STEMCELL Technologies). Half of the media was replaced on Day 2. On Days 4 and 6, half of the media was replaced with GMEM KSR media (GMEM, 20% KSR, 1×NEAA (Gibco), 1× pyruvate (Gibco), 1× β-mercaptoethanol, 1× antibiotic-antimycotic) supplemented with 5 mM SB-431542, 3 mM IWR1e, 2.5 mM cyclopamine (STEMCELL Technologies) and 2 mM thiazovivin. On Day 8, half of the media was replaced with GMEM KSR media supplemented with 5 μM SB-431542, 3 mM IWR1e, and 2.5 μM cyclopamine. On Days 10, 12, 14, and 16, half the media was replaced with GMEM KSR media supplemented with 5 mM SB-431542 and 3 mM IWR1e. On Days 18 and 20, half of the media was replaced with CBO N2 media (DMEM: F12, 1× chemically defined lipid concentrate (Life Technologies), 1×N2 supplement (Gibco) and 100× antibiotic-antimycotic) supplemented with 1×B27 supplement without vitamin A (Gibco), 10 ng/mL bFGF

(STEMCELL Technologies), and 10 ng/mL EGF (Peprotech). On Day 22, organoids were transferred to a magnetic stir bioreactor (ABLE Corporation) in CBO N2 media supplemented with 1×B27 supplement without vitamin A, 10 ng/mL bFGF, and 10 ng/ml EGF (Peprotech), and agitated at 4 rpm. Half the media was replaced on Days 24, 26, and 28. On Day 30, the media was changed to CBO FBS media (DMEM:F12, 1× chemically defined lipid concentrate (Life Technologies), 1×N2 supplement, 10% ES-FBS, 5 μg/mL heparin and 1X antibiotic-antimycotic) supplemented with 1×B27 supplement without vitamin A. Complete media was replaced every 4 days. On Days 42 and 46, the media was changed to CBO FBS media supplemented with 1×B27 supplement without vitamin A, 10 ng/mL BDNF (Peprotech), and 10 ng/mL GDNF (Peprotech). Starting Day 50, the media was changed to BRAINPHYS® media (STEMCELL technologies) supplemented with 1×N2 supplement, 1×B27 supplement without vitamin A, 10 ng/mL BDNF, and 10 ng/mL GDNF. Complete media was replaced every 4 days. Starting at Day 35, large cortical organoids were pinched into two halves by using a pair of sterile forceps; this was repeated once every 5-7 days to avoid large necrotic centers.

Postmortem Human Samples. Material from the superior temporal gyrus of eight subjects with WBS (age 17-69 years) and eight age- and sex-matched control subjects was obtained from the NIH Neurobiobank at the University of Maryland. Three fresh-frozen samples each from the WBS and control subjects were used for western blot analysis. Five fixed samples each from the WBS and control subjects were received, and four were of sufficiently good quality to be used for immunohistochemical analysis.

Statistics. Statistics were calculated using EXCEL® (Microsoft), SIGMAPLOT® (Systat), Prism (Graphpad), R, or Python. Graphs are represented as bars showing mean±SEM, with overlaid dots showing individual measurements, and as box or violin plots. Statistical comparisons used are noted. Unless otherwise noted, distributions were tested for normality (Shapiro-Wilk test) and equal variance (Brown-Forsythe test). If the distribution passed, a paired or unpaired t-test was performed. If it failed, a rank sum test or signed rank test was performed. To compare more than two distributions, one-way, two-way or repeated measures (RM) ANOVAs were performed. To compare cumulative distributions, a Kolmogorov-Smirnov test was used. Significance was designated as P<0.05.

### Example 2: WBS Mice Have Enhanced Innate Pitch-Discrimination

To determine whether WBS mice had altered pitch discrimination, 6- to 12-week-old wild-type (WT) mice were compared to CD^{+/-} mouse models of WBS, which carry a microdeletion spanning from Fkbp6 to Gtf2i (Segura-Puimedon et al. (2014) Hum. Mol. Genet. 23:6481-6494) using a test based on pre-pulse inhibition (PPI) of the auditory startle response (ASR). Frequency discrimination-driven PPI relies on an innate behavior-ASR to loud noise (Aizenberg & Geffen (2013) Nat. Neurosci. 16:994-996; Aizenberg et al. (2015) PLOS Biol. 13:e1002308; Clause et al. (2011) J. Neurosci. Methods 200:63-67). PPI of the ASR is directly proportional to the shift between the background frequency and the frequency of pre-pulse tones, with greater PPI indicating an animal detecting the shift in frequency more robustly. PPI was increasingly enhanced in both genotypes with larger shifts between background frequency and pre-pulse frequencies. However, CD^{+/-} mice exhibited significantly greater PPI at pre-pulse frequencies closer to background than did WT littermates. The frequency-discrimination threshold (FDT) was measured in WT and CD^{+/-} mice as a reliable, accurate measure of innate frequency-discrimination acuity (Aizenberg & Geffen (2013) Nat. Neurosci. 16:994-996; Aizenberg et al. (2015) PLOS Biol. 13: e1002308; Clause et al. (2011) J. Neurosci. Methods 200:63-67). FDT in CD^{+/-} mice was, on average, ˜50% lower than that in WT mice (WT FDT 6.01±1.35; CD^{+/-} FDT 2.96±0.52, P=0.019). CD^{+/-} male and female mice were equally affected (two-way ANOVA, P_{genotype}=0.03, Pₛₑₓ=0.8). This indicates that WBS mice can discriminate acoustic frequencies substantially better than WT mice, indicating that WBS mice have innate pitch-discrimination hyperacuity.

ASR alone did not differ between WT and CD^{+/-} mice, suggesting that auditory hyperacuity was not due to changes in the startle reflex behavior. No detectable difference was observed between genotypes in the auditory brainstem response (ABR) test, which measures the initial steps in sound processing, including cochlear transduction and brainstem nuclei responsiveness (Ingham et al. (2011) Curr. Protoc. Mouse Biol. 1:279-287). There was also no difference in the threshold sound intensity needed to elicit an ABR between WT and CD^{+/-} littermates across all frequencies tested, including those near the background frequency used in the PPI experiments. This demonstrates that CD^{+/-} mice have intact peripheral hearing at the ages used in these experiments and indicates that auditory hyperacuity in these WBS mutants arises at the central parts of the auditory system.

### Example 3: Increased Cortical Inhibition in the ACx of WBS Mice

Previous work has implicated synaptic interactions in the primary ACx as contributing to frequency-discrimination acuity (Aizenberg et al. (2015) PLOS Biol. 13: e1002308). Therefore, the cellular and circuit properties of neurons were compared in WT and CD^{+/-} mice by performing whole-cell recordings in acute brain slices containing the ventral division of the medial geniculate (MGv, auditory thalamus), the primary ACx, and functional connections between these regions (Blundon et al. (2011) J. Neurosci. 31:16012-16025; Chun et al. (2013) J. Neurosci. 33:7345-7357; Cruikshank et al. (2002) J. Neurophysiol. 87:361-384).

It was found that spontaneous excitatory activity was significantly reduced in excitatory neurons in the ACx but not in the MGv in WBS models. Spontaneous excitatory synaptic currents (sEPSCs) were recorded in Layer (L) 4 (thalamorecipient) excitatory in neurons the ACx, the majority of which are pyramidal neurons (Richardson et al. (2009) J. Neurosci. 29:6406-6417; Smith & Populin (2001) J. Comp. Neurol. 436:508-519). It was observed that sEPSCs were significantly less frequent in CD^{+/-} mice compared to WT littermates. In contrast, sEPSCs recorded in thalamic MGv excitatory (relay) neurons did not differ in frequency between the genotypes. The amplitude of SEPSCs was preserved in CD^{+/-} mice in both regions.

Reduced sEPSC frequency in the CD^{+/-} ACx was not caused by reduced input from the thalamus. Evoked postsynaptic currents recorded in L4 cortical excitatory neurons and elicited by stimulating ascending thalamocortical axons did not differ in amplitude or paired-pulse ratio between WT and CD^{+/-} mice.

Reduced SEPSC frequency in the CD^{+/-} ACx was also not caused by changes in excitability of cortical or thalamic excitatory neurons. No differences were found between WT and CD^{+/-} mice in terms of threshold or number of action potentials elicited in response to current steps or ramps. In addition, no difference was observed in the resting membrane potential or input resistance, and no difference in the properties of individual action potentials elicited by current injection in ACx L4 excitatory was cells found. Similar characterization of excitatory (relay) neurons in the MGv also showed no difference in intrinsic and action potential properties between WT and CD^{+/-} mice. Together, these results imply abnormality in the local synaptic circuitry in the ACx but not in the thalamus or thalamocortical projections.

It was subsequently determined whether the reduced SEPSC frequency in the CD^{+/-} ACx was due to elevated cortical inhibition. The difference in sEPSC frequencies recorded from ACx excitatory neurons was abolished after application of either the GABAA receptor antagonist picrotoxin (PTX) or the voltage-gated Na⁺ channel blocker tetrodotoxin (TTX). The amplitude of EPSCs was not altered between WT and CD^{+/-} pyramidal neurons under either condition, indicating that the postsynaptic glutamate receptors at excitatory synapses are unchanged between genotypes. Together, these results indicate that the decreased sEPSC frequency observed in CD^{+/-} mice was not a direct property of the presynaptic glutamatergic inputs but rather was a consequence of increased inhibition in the ACx circuit.

Consistent with this notion, a higher frequency of miniature inhibitory postsynaptic currents (mIPSCs) was observed in pyramidal neurons of the CD^{+/-} ACx. The amplitude of mIPSCs did not differ between WT and CD^{+/-} mice. Together, these results indicate that in WBS mice cortical excitatory neurons receive similar excitatory inputs but stronger inhibitory inputs. With the cortical network active, the net result was reduced spontaneous excitatory synaptic activity in the ACx.

### Example 4: Hyperexcitability of Inhibitory Interneurons (But Not Excitatory Neurons) in the ACx of WBS Mice

It was subsequently demonstrated that the source of elevated inhibition in the ACx of WBS mice was the mechanism of auditory Previous hyperacuity. work showed that optogenetic stimulation of PV⁺ inhibitory interneurons in the ACx improves frequency discrimination in mice (Aizenberg et al. (2015) PLOS Biol. 13:e1002308). Recordings were taken from L4 fast-spiking (FS) interneurons in auditory thalamocortical slices, most of which express parvalbumin and include the major subclass of cortical interneurons (Scala et al. (2019) Nat. Commun. 2019 101 10:1-12; Tremblay et al. (2016) Neuron 91:260-292). PV⁺ neurons were targeted in auditory thalamocortical slices either by using PV^{Cre}; Ai14; CD^{+/-} and PV^{Cre}; Ai14; WT mice, which express tdTomato in PV⁺ cells, or by assessing their soma size, shape, location, and subsequently verifying their physiological properties during recording in CD^{+/-} and WT mice. Using current-clamp mode and recording the membrane voltage of FS interneurons, it was found that the same depolarizing currents elicited more action potentials in CD^{+/-} interneurons than in WT interneurons. This was evident at smaller (100 pA) but not larger (250 pA) currents, indicating that the threshold for eliciting action potentials (rheobase) was reduced in CD^{+/-} mice. To measure rheobase, ramps of increasing current were delivered in the presence of kynurenic acid and PTX to block ionotropic glutamate receptors and GABA receptors, respectively. Action potentials were evoked at significantly lower currents in CD^{+/-} interneurons than in WT cells, indicating that mutant interneurons have a reduced rheobase. These results indicate that inhibitory FS interneurons in the ACx of WBS mice are hyperexcitable.

The persistence of this hyperexcitability phenotype in the presence of synaptic blockers implied that an intrinsic property of FS interneurons accounts for their hyperexcitability. However, there was no significant difference in the resting membrane potential or input resistance of these neurons between WT and CD^{+/-} mice, suggesting that their hyperexcitability originated from the active properties of interneurons, such as voltage-dependent conductances.

Changes were screened for in known voltage-gated channels by recording in voltage-clamp mode from FS cells. No difference was found between WT and CD^{+/-} FS interneurons in terms of the amplitude of voltage-gated Na⁺ currents, voltage-gated K⁺ currents, or hyperpolarization-activated cationic currents (Iₕ) conducted by hyperpolarization-activated cyclic nucleotide-gated channels. However, a difference was found between genotypes when a protocol commonly used to isolate voltage-gated Ca²⁺ currents (Olson et al. (2005) J. Neurosci. 25:1050-1062) was employed. TTX was added to the artificial cerebral spinal fluid (ACSF) to block Na⁺ channels, Cs⁺ and TEA (tetraethylammonium) were added to the intracellular solution to block K⁺ channels, and 3 mM Ba²⁺ was substituted for Ca²⁺ in the ACSF to limit Ca²⁺-dependent channel inactivation. When depolarizing voltage steps were delivered to FS interneurons, an inward voltage-gated current activated at more hyperpolarized voltages was observed in CD^{+/-} mice than in WT mice. A voltage ramp was then delivered to precisely measure the threshold at which the inward conductance was activated and an ˜2.5 mV lower threshold was found in CD^{+/-} mice than in WT mice. The total inward current generated by the voltage ramp was not significantly different. This additional inward current component in CD^{+/-} interneurons was activated at voltages close to the threshold for action potential generation; therefore, it may account for the extra activity-dependent depolarization and hyperexcitability of ACx interneurons in WBS mice.

### Example 5: Chemogenetic Inhibition of ACx Interneuron Hyperexcitability Rescues Pitch-Discrimination Hyperacuity in CD^{+/-} Mice

It was posited that if interneuron hyperexcitability underlies the pitch-discrimination hyperacuity in CD^{+/-} mice, then decreasing interneuron activity in the ACx should rescue the phenotype. To reduce the excitability of ACx interneurons, a chemogenetic approach was used, wherein the designer receptor exclusively activated by designer drug (DREADD) hM4Di was expressed, which hyperpolarized neurons after activation by its synthetic inert ligand Compound 21 (C21) (Thompson et al. (2018) ACS Pharmacol. Transl. Sci. 1:61-72). To express hM4Di specifically in ACx interneurons, recombinant AAVs (rAAVs) encoding Cre-dependent hM4Di (rAAV-hSyn-DIO-hM4Di-IRES-mCitrine) was injected bilaterally into the ACx of Gad2^{Cre}; WT mice and Gad2^{Cre}; CD^{+/-} mice (Gad2, glutamic acid decarboxylase 2 locus). Gad2^{Cre} mice express Cre recombinase in most interneurons (Ledri et al. (2014) J. Neurosci. 34:3364-3377). This was validated by using immunochemistry in Gad2^{Cre}; Ai14 mice (the Ai14 strain is a reporter mouse expressing tdTomato in a Cre-dependent manner), which showed colocalization of tdTomato and GABA It was also signals in putative GABAergic interneurons.

established that rAAV injection sites were localized to the ACx. Recording from hM4Di-expressing (mCitrine+) cells in the ACx from acute cortical slices confirmed that bath application of C21 decreased the number of action potentials elicited by depolarizing current injections in cortical FS interneurons.

It was subsequently determined whether chemogenetic reduction of interneuron excitability eliminated pitch-discrimination hyperacuity in WBS mice by returning it to the WT level. In these experiments, either vehicle or C21 was injected intraperitoneally into Gad2^{Cre}; WT or Gad2^{Cre}; CD^{+/-} mice that bilaterally expressed hM4Di in interneurons of the ACx and about 30 minutes later, pitch-discrimination was tested using PPI, as previously done. Several days later, the same group of animals was injected with the opposite drug (C21 or vehicle) and pitch discrimination was tested again. Injections were randomized using a within-subjects, counterbalanced design to reduce a possible effect of treatment order. Gad2^{Cre}; CD^{+/-} mice significantly increased FDT (or reduced pitch-discrimination acuity) after injection of C21 compared to vehicle injections. In contrast, FDT in Gad2^{Cre}; WT mice was not different between C21 and vehicle injections. FDT in Gad2^{Cre}; CD^{+/-} mice treated with C21 returned to WT levels, thereby demonstrating complete rescue. ASR was not affected by C21 in either genotype. Together, these data showed that the ACx is one of the sites necessary for pitch-discrimination hyperacuity behavior in WBS mice. These results also pointed to hyperexcitability of inhibitory interneurons as the cellular mechanism of pitch-discrimination hyperacuity in WBS.

### Example 6: Improved Frequency Coding by the ACx in WBS Mice

To examine how changes in ACx circuitry in WBS mice influence the encoding of frequency information, sound-evoked activity was measured in the ACx of awake mice. Two-photon imaging of sound-evoked activity was performed simultaneously in hundreds of individual excitatory neurons expressing the genetically encoded fluorescent Ca²⁺ indicator GCaMP6f (Chen et al. (2013) Nature 499:295-300; Romano et al. (2015) Neuron 85:1070-1085). To selectively express GCaMP6f in L4 excitatory neurons, Ai93 mice (TIGRE-Ins-TRE-LSL-GCaMP6f) (Madisen et al. (2015) Neuron 85:942-958) were crossed with CamKIIα^{tTA} mice (excitatory neurons specificity) and with Scnn1a^{Cre} mice (L4 specificity). The resultant transgenic mice were referred to as GCaMP6f^{E×N-L4} mice. Those mice were then crossed with CD^{+/-} mice and a cranial window was installed in each offspring. The resultant mice expressed GCaMP6f in L4 excitatory neurons throughout the ACx. Several weeks after surgery, tones were delivered at multiple frequencies and intensities (attenuations) in a pseudo-random order to awake GCaMP6f^{E×N-L4}; WT mice and GCaMP6f^{E×N-L4}; CD^{+/-} mice walking on a rotating disk and the activity of tone-evoked changes in GCaMP6f fluorescence responses were analyzed in individual neurons. In total, data from 7130 cells in 36 mice (GCaMP6f^{E×N-L4}; WT; 5726 cells, 30 mice; GCaMP6f^{E×N-L4}; CD^{+/-}; 1404 cells, 6 mice) were collected.

The cell bodies of active LA excitatory neurons were identified and segmented, their Ca²⁺ responses were measured (Blundon et al. (2017) Science 356:1352-1356) and deconvolved (Friedrich et al. (2017) PLOS Comput. Biol. 13: e1005423) to categorize each neuron's receptive field and best frequency as measures of frequency tuning. As reported previously (Kanold et al. (2014) Trends. Neurosci. 37:502-510), frequency tuning of individual cells was heterogeneous in the ACx. It has been proposed that frequency encoding, as it relates to perception, most likely involves the activity of groups of neurons (Downer et al. (2021) J. Neurosci. 41:7561-7577; See et al. (2018) Elife 7: e35587). To determine the frequency-coding capacity of excitatory neurons, machine learning was used to decode the occurrence of tones and their frequency from the deconvolved Ca²⁺ responses of all imaged neurons from each mouse. Linear decoders were trained to predict the occurrence of the tones and their frequencies. The linear decoder for tone prediction performed equally well (>80%) in GCaMP6f^{E×N-L4}; WT mice and GCaMP6f^{E×N-L4}; CD^{+/-} mice. The frequency decoder was used that predicts the specific frequency of a tone presented based on the activity of L4 neurons in the ACx. This decoder more accurately predicted the frequencies in CD^{+/-} neurons than in WT neurons.

To understand why the frequency decoder was more accurate in CD^{+/-} mice than WT littermates and identify the elements responsible for the difference, the decoder was modified in several ways. First, the window of analysis was restricted from 400 ms to 100 ms after the tone presentation. Under those conditions, the frequency decoder performed equally well between WT and CD^{+/-} mice, suggesting that later temporal components of the sound-evoked responses in CD^{+/-} mice provide additional frequency information. In fact, the responses in CD^{+/-} neurons were more sustained, as evidenced by the significantly longer temporal autocorrelation range of individual responses in CD^{+/-} neurons than in WT neurons. Additionally, to rule out the possibility that differences in the total number of cells or in the fraction of tone-responsive cells help improve frequency coding, neuronal populations were randomly selected, which matched those variables between WT and CD^{+/-} mice. The improved frequency decoder accuracy persisted in CD^{+/-} mice, Together, these results indicated that prolonged temporal components of the tone-evoked neural responses in the ACx of WBS mice enable them to better encode frequency information. These results also indicated that improved frequency encoding contributes to innately enhanced pitch-discrimination acuity in WBS mice.

### Example 7: Haploinsufficiency of the WBS Gene Gtf2ird1 Recapitulates the Pitch-Discrimination Hyperacuity Phenotype of CD^{+/-} Mice

It was subsequently determined which WBS gene(s) causes the auditory-hyperacuity phenotype of WBS mice. This analysis took advantage of mice that carry smaller microdeletions within the region commonly deleted in persons with WBS. Specifically, mice with either a proximal deletion (PD) or distal deletion (DD) were used (Li et al., 2009). The PD spanned Limk1-Gtf2i, and the DD spanned Trim50-Limk1. Together, these two microdeletions encompassed the entire CD microdeletion. The phenotype of PD^{+/-} mice was similar to that of CD^{+/-} mice, and DD^{+/-} mice performed at the WT level. Like CD^{+/-} mice, PD^{+/-} mice had an FDT that was ˜50% lower than that of WT littermates (WT FDT 8.25±1.52; PD^{+/-} FDT 4.02±0.98, P=0.03). In contrast, the FDT in DD^{+/-} mice and that in WT mice were similar (WT FDT 5.56±1.23; PD^{+/-} FDT 7.72±1.13, P=0.23). These results indicated that the causal gene(s) is contained within the PD region of the WBS microdeletion.

Of the genes in deleted the PD region, haploinsufficiency of genes encoding transcription factors, Gtf2ird1 (Howard et al. (2012) Neurobiol. Dis. 45:913-922; Proulx et al. (2010) J. Neurodev. Disord. 2:99-108; Schneider et al. (2012) Behav. Brain Res. 233:458-473; Young et al. (2008) Genes Brain Behav. 7:224-234) and Gtf2i (Barak et al. (2019) Nat. Neurosci. 22:700-708), had been implicated in cognitive abnormalities. Human subjects with partial deletions of the WBS region that include GTF2IRD1 and GTF2I have cognitive deficits similar to those of persons with WBS (Broadbent et al. (2014) J. Neurodev. Disord. 6:18; Tassabehji et al. (2005) Science 310:1184-1187); conversely, individuals with partial deletions that exclude these genes have more preserved cognitive function (Antonell et al. (2010) J. Med. Genet. 47:312-320; van Hagen et al. (2007) Neurobiol. Dis. 26:112-124; Hirota et al. (2003) Genet. Med. 5:311-321).

Because the heterozygous or homozygous disruption of the Gtf2ird1 gene in mice has been implicated in the neurological abnormalities associated with WBS, this gene was further investigated. It was observed that like CD^{+/-} mice and PD^{+/-} mice, Gtf2ird1^{+/-} and Gtf2ird1^{-/-} mice performed significantly better than WT littermates in the pitch-discrimination behavioral test. Specifically, the FDT was reduced by ˜48% and ˜44% in Gtf2ird1^{+/-} and Gtf2ird1^{-/-} mutants, respectively, compared to WT mice (WT FDT 6.90±0.89; Gtf2ird1^{+/-} FDT 3.63±0.46; Gtf2ird1^{-/-} EDT 3.94±0.59; F₂=6.36, P=0.004). The ASR was not affected in the PD^{+/-}, DD^{+/-} Gtf2ird1^{+/-}, or Gtf2ird1^{-/-} mutants. Together, these results indicate that Gtf2ird1 is the critical gene, the hemizygous deletion of which causes pitch-discrimination hyperacuity in WBS mouse models.

### Example 8: Gtf2ird1 Haploinsufficiency Leads to Downregulation of Vipr1 in Mice

GTF2IRD1 is a transcription factor with many downstream gene targets (Kopp et al. (2020) Hum. Mol. Genet. 29:1498-1519) that could function in pitch-discrimination hyperacuity. Because the critical cellular mechanism of auditory hyperacuity is hyperexcitability of cortical interneurons (but not excitatory cells), differentially regulated genes were screened for in cortical interneurons from Gtf2ird1^{-/-} mice. To isolate interneurons, Gtf2ird1^{-/-} mice were generated that express tdTomato under control of the interneuron-selective Gad2 promoter (Gad2^{Cre}; Ai14; Gtf2ird1^{-/-} mice) and tdTomato⁺ cells were sorted from the cortex of Gad2^{Cre}; Ai14; Gtf2ird1^{+/+} mice and Gad2^{Cre}; Ai14; Gtf2ird1^{-/-} mice. RNA-seq analysis revealed a number of genes significantly differentially expressed in Gtf2ird1^{-/-} vs. WT interneurons. Initial focus was placed on the Vipr1 gene, which encodes the G-protein-coupled vasoactive intestinal polypeptide receptor 1 (VIPR1, also known as VPAC1), as it was significantly downregulated in Gtf2ird1^{-/-} interneurons. VIPR1 affects the activity of multiple voltage-gated channels (Gherghina et al. (2017) Brain Res. 1657:297-303; Hayashi et al. (2002) Bull. Tokyo Dent. Coll. 43:31-39; Tang et al. (2019) Oncogene 38:3946-3961; Zhu & Ikeda (1994) Neuron 13:657-669); therefore, it could mediate the hyperexcitability phenotype observed in CD^{+/-} interneurons. Using RT-qPCR, it was determined that Gtf2ird1 and Vipr1 transcripts were also decreased in GAD2⁺ interneurons from WBS mice. Specifically, the levels of Gtf2ird1 and Vipr1 were reduced by approximately half in Gad2^{Cre}; Ai14; CD^{+/-} mice compared to Gad2^{Cre}; Ai14; WT mice.

### Example 9: VIPR1 is Downregulated in the ACx Interneurons of Individuals With WBS and in Cortical Organoids Derived From hiPSCs With an Isogenic WBS Microdeletion

To examine whether VIPR1 downregulation occurs in humans with WBS, postmortem samples of brain tissue were obtained from the superior temporal gyrus (containing the ACx) patients with WBS. The level of VIPR1 was significantly reduced in the whole-brain lysate of human subjects with WBS compared to healthy controls, based on western blot analysis. To examine the levels of VIPR1 expression specifically in interneurons, cortical sections were immunolabelled with VIPR1 and the FS interneuron marker PV and the intensity of VIPR1 in PV⁺ cells was quantified. Significantly lower intensity VIPR1 staining was observed in PV⁺ interneurons from WBS brains compared to controls, with no change in the size or number of PV⁺ neurons.

VIPR1 expression was also reduced in cortical organoids generated from human hiPSCs with hemizygous microdeletion of the WBS region. To generate mutant organoids, CRIPSPR/Cas9 was used to introduce the 1.57-Mb microdeletion encompassing the NSUN5-GTF2IRD2 genomic region in hiPSCs from a healthy individual. Cortical organoids were generated and gene expression was compared between NSUN5-GTF2IRD2^{+/-} organoids and isogenic control organoids by bulk RNA-seq. The expression of almost all detected protein-coding WBS genes within the NSUN5-GTF2IRD2 microdeletion was significantly reduced in mutant organoids, suggesting that NSUN5-GTF2IRD2^{+/-} cortical organoids can model WBS at the transcriptional level. Expression of VIPR1 was also significantly reduced in NSUN5-GTF2IRD2^{+/-} cortical organoids compared to isogenic controls.

### Example 10: Acute Inhibition of VIPR1 Mimics WBS Interneuron Phenotypes by Causing Hyperexcitability and Lowering the Threshold of Inward Voltage-Gated Current in ACx Interneurons

To identify how the reduced level of VIPR1 affects the function of FS interneurons in WBS mice, the VIPR1-specific antagonist PG 97-269 was used. Blocking VIPR1 increased the excitability of FS interneurons in WT cortex, which significantly lowered the threshold for action potential induction in response to a current ramp, thereby mimicking the CD^{+/-} phenotype. The difference between rheobase before and after application of PG 97-269 (PG 97-269 rheobase shift) was significantly less than 0 in WT mice (P=0.01). In the CD^{+/-} cortex, interneurons were already hyperexcitable, as previously observed, and the addition of PG 97-269 had no additional effect on excitability. Therefore, the PG 07-269 rheobase shift in CD^{+/-} interneurons was significantly higher than that in WT interneurons (P<0.05) and not significantly different from 0 (P=0.11). Similarly, in WT mice, applying PG 97-269 mimicked the CD^{+/-} phenotype by shifting the threshold of the inward voltage-gated current in ACx interneurons to more hyperpolarized potentials. The difference between the inward current thresholds in the presence of PG 97-269 and vehicle (PG 97-269 threshold shift) was 2.1 mV±0.4 mV, which was similar to the threshold difference between CD^{+/-} and WT interneurons. The PG 97-269 threshold shift for inward voltage-gated current was significantly smaller in CD^{+/-} interneurons than WT interneurons (0.6 mV±0.2 mV, P<0.05). These results indicated that in WT cortex tonic VIPR1 activity decreases the excitability of FS interneurons, and this activity is absent in CD^{+/-} mice, possibly as a consequence of decreased receptor levels. The VIPR1-specific agonist [Ala^{11,22,28}]-VIP had no significant effect on WT or CD^{+/-} interneurons, indicating that an endogenous VIPR1 ligand is present at high enough concentration to saturate VIPR1 receptors in brain slices. Together, these results demonstrated that hyperexcitability of WBS interneurons in the ACx is caused by reduced VIPR1 signaling.

### Example 11: Gtf2ird1 Deletion Causes Interneuron Hyperexcitability and Lowers the Threshold of Inward Voltage-Gated Current in ACx Interneurons

The behavioral studies demonstrated that Gtf2ird1 is the crucial gene that leads to auditory hyperacuity in WBS mice. It was subsequently determined whether the cellular phenotypes observed in CD^{+/-} interneurons are also present in mice with a deletion of Gtf2ird1. Hyperexcitability of FS interneurons was observed in the ACx of Gtf2ird1^{+/-} mice and Gtf2ird1^{-/-} mice. Rheobase measured in these mutants did not differ from that in interneurons of CD^{+/-} mice (P=0.503). There was either a diminished or no effect of PG 97-269 on the excitability of FS interneurons in the ACx of Gtf2ird1^{+/-} mice and Gtf2ird1^{-/-} mice compared to WT mice. The PG 97-269 rheobase shift in FS interneurons did not significantly differ from 0 in Gtf2ird1^{-/-} mice (P=0.056), although it did differ from 0 in Gtf2ird1+/− mice (P<0.001). However, the PG 97-269 rheobase shift was significantly more pronounced in WT interneurons than in Gtf2ird1-deficient interneurons (P<0.05).

Likewise, the inward voltage-gated current-activation threshold in Gtf2ird1^{+/-} and Gtf2ird1^{-/-} cortical interneurons was shifted compared to that in WT interneurons and was less sensitive to PG 97-269, similar to results from CD^{+/-} mice. Threshold for inward current activation in Gtf2ird1^{+/-} and Gtf2ird1^{-/-} cortical interneurons was shifted toward more negative values by ˜3.5-4.0 mV compared to that in WT interneurons. The PG 97-269 threshold shift was also significantly reduced (P<0.05) in Gtf2ird1^{+/-} and Gtf2ird1^{-/-} cortical interneurons compared to WT interneurons. The consistency of the cellular phenotypes between Gtf2ird1-deficient mutants and CD^{+/-}0 mice bolsters the evidence that Gtf2ird1 is the critical WBS gene that regulates the ACx interneuron excitability and, in turn, pitch-discrimination acuity in WBS mouse models via downregulation of VIPR1.

### Example 12: Vipr1 Reduction in Interneurons is Necessary and Sufficient for the Pitch-Discrimination Hyperacuity and Interneuron Hyperexcitability in WBS Mice

To determine whether VIPR1 reduction in FS

interneurons underlies (i.e., necessary and sufficient) pitch-discrimination hyperacuity in WBS mice, an attempt was made to rescue mimic or the pitch discrimination WBS hyperacuity and interneuron hyperexcitability by genetically reducing or replenishing the VIPR1 level in ACx interneurons in WT mice or CD^{+/-} (or Gtf2ird^{+/-}) mice, respectively.

The in vivo expression of Vipr1 was chronically reduced by generating a conditional knockout (cKO) mouse in which Vipr1 was deleted only in interneurons. To this end, a mouse with the floxed exon 2 in the Vipr1 gene (Vipr1^{fl/+} mice) was generated. These mice were then crossed with Gad2^{Cre} mice to generate mice with conditional deletion of Vipr1 in GAD2⁺ cells (Gad2^{Cre}; Vipr1^{fl/+} mice). The Vipr1 transcript was reduced in a dose-dependent manner in the cortex of Gad2^{Cre}; Vipr1^{fl/+} mice and Gad2^{Cre}; Vipr1^{fl/fl} mice compared to WT (Gad2^{Cre}; Vipr1^{+/+}) littermates.

VIPR1-deficient FS interneurons displayed a hyperexcitability phenotype like that of CD^{+/-} mice. Rheobase was significantly reduced in FS interneurons of Gad2^{Cre}; Vipr1^{fl/+} mice and Gad2^{Cre}; Vipr1^{fl/fl} mice compared to that in WT littermates. Behaviorally, Gad2^{Cre}; Vipr1^{fl/+} mice and Gad2^{Cre}; Vipr1^{fl/fl} mice had a normal acoustic startle response but showed improved frequency discrimination comparable to that of CD^{+/-}0 mice. These data indicated that chronically decreased expression of Vipr1 only in interneurons, which is most likely representative of the WBS condition, is sufficient to mimic the behavioral and cellular phenotypes of WBS mice.

To determine whether Vipr1 depletion in FS interneurons is necessary for FS interneuron hyperexcitability and pitch-discrimination hyperacuity in WBS mice, the WBS phenotypes were restored by increasing the expression of Vipr1 in FS interneurons. For this, three different strategies were used. First, rAAVs were used, which expressed Vipr1 and GFP under control of the human form of the Dlx5/6 enhancer (hDlx) (AAV-hDlx-Vipr1-GFP), which restricts the expression to GABAergic interneurons (Dimidschstein et al. (2016) Nat. Neurosci. 19:1743-1749). A high degree of co-localization was observed between GFP and tdTomato fluorescence in GAD2⁺ cells when AAV-hDlx-Vipr1-GFP was injected into the ACx of Gad2^{Cre}; Ai14 mice. This strategy was used to restore Vipr1 expression in FS interneurons in Gtf2ird1^{+/-}0 mice. After bilateral injections of AAV-hDlx-Vipr1-GFP (or AAV-hDIx-GFP as control) into the ACx, recordings from GFP⁺ FS interneurons in thalamocortical slices were taken. WT interneurons showed no difference in excitability between the two conditions; however, Gtf2ird1^{+/-}0 FS interneurons with overexpressed Vipr1 (but not GFP), showed significantly reduced excitability (increased rheobase) compared to the WT level. Behaviorally, none of the viruses altered the ASR, but AAV-hDlx-Vipr1-GFP significantly increased FDT (reducing pitch-discrimination hyperacuity) in Gtf2ird1^{+/-} mice but not in WT mice, though AAV-hDlx-GFP expression alone did not. These results indicated that Vipr1 replenishment in ACx interneurons rescued the cellular and pitch-discrimination acuity phenotypes in Gtf2ird1^{+/-} mice.

An attempt was then made to rescue the cellular and behavioral phenotypes in CD^{+/-} models of WBS by using two alternative strategies (viral and transgenic) to overexpress Vipr1 in interneurons. Using a viral strategy, rAAVs encoding Cre-dependent Vipr1 and GFP (AAV-CAG-Flex-Vipr1-GFP) or AAV-CAG-Flex-GFP control were injected into the ACx of Gad2^{Cre} mice crossed with CD^{+/-} mice. Recording from GFP⁺ cells showed no difference in excitability between WT Vipr1-overexpressing FS interneurons in WT mice. However, in CD^{+/-}0 mice, increasing Vipr1 expression elevated rheobase (reducing excitability) to the WT level, while FS interneuron hyperexcitability was maintained with control GFP virus. Behaviorally, none of the viral injections altered the ASR, but Vipr1 overexpression significantly increased the FDT (reducing pitch-discrimination hyperacuity) in CD^{+/-} mice but not in WT mice, though GFP expression alone did not.

Finally, using a transgenic strategy, a mutant mouse was generated with conditional overexpression of Vipr1 in interneurons (Vipr1^{cOE} mice). Vipr1^{cOE} mice were produced and later validated by generating a transgenic mouse expressing CAG-LSL-Vipr1-IRES-GFP and crossing this mouse with Gad2^{Cre} mice. An additional cross with CD^{+/-} mice generated Vipr1^{cOE}; CD^{+/-} and Vipr1^{cOE}; WT mice. Transgenic overexpression of Vipr1 in interneurons did not affect ASR in WT or CD^{+/-} mutants. Nor did it affect the rheobase or pitch discrimination in WT mice. However, Vipr1^{cOE}; CD^{+/-} mice performed at the WT levels both cellularly and behaviorally. Overexpression of Vipr1 in interneurons of CD^{+/-} mice rescued the rheobase phenotype in ACx interneurons and the pitch discrimination phenotype.

Together these data demonstrate that reduced Vipr1 expression in ACx interneurons is necessary and sufficient for both the behavioral and cellular phenotypes of WBS mice.

1. A method for increasing auditory cortex interneuron excitability comprising contacting an auditory cortex interneuron with a Vasoactive Intestinal Peptide Receptor 1 (VIPR1) inhibitor thereby increasing the excitability of the auditory cortex interneuron.

2. The method of claim 1, wherein the VIPR1 inhibitor decreases the activity of VIPR1.

3. The method of claim 2, wherein the VIPR1 inhibitor is a VIPR1 peptide antagonist.

4. The method of claim 1, wherein the VIPR1 inhibitor decreases the expression of VIPR1.

5. The method of claim 4, wherein the VIPR1 inhibitor is a VIPR1 inhibitory RNA molecule.

6. A method for improving auditory perception in a subject comprising administering to a subject in need thereof a Vasoactive Intestinal Peptide Receptor 1 (VIPR1) inhibitor thereby improving the subject's auditory perception.

7. The method of claim 6, wherein the VIPR1 inhibitor decreases the activity of VIPR1.

8. The method of claim 7, wherein the VIPR1 inhibitor is a VIPR1 peptide antagonist.

9. The method of claim 6, wherein the VIPR1 inhibitor decreases the expression of VIPR1.

10. The method of claim 9, wherein the VIPR1 inhibitor is a VIPR1 inhibitory RNA molecule.

11. The method of claim 6, wherein the auditory perception comprises pitch-discrimination acuity.

12. The method of claim 6, wherein the subject is a musician, linguist, or has amusia.
